SEQUENCE LISTING (88 sequences)

SEQ ID NO: 1 - RNA, 21 nt, Artificial Sequence
  Construct
  misc_feature (1)..(1): N = A, G or U
  misc_feature (17)..(17): N = G or U
  misc_feature (18)..(18): N = A or G
  misc_feature (21)..(21): N = A, C, G or U
ngaaucuugaugaugcnncan

SEQ ID NO: 2 - RNA, 21 nt, Malus domestica
agaaucuugaugaugcugcau

SEQ ID NO: 3 - RNA, 21 nt, Pyrus bretschneideri
agaaucuugaugaugcugcau

SEQ ID NO: 4 - RNA, 21 nt, Pyrus communis
agaaucuugaugaugcugcau

SEQ ID NO: 5 - RNA, 21 nt, Prunus persica
agaaucuugaugaugcugcau

SEQ ID NO: 6 - RNA, 20 nt, Citrus sinensis
agaaucuugaugaugcugca

SEQ ID NO: 7 - RNA, 21 nt, Vitis vinifera
ugaaucuugaugaugcuacau

SEQ ID NO: 8 - RNA, 21 nt, Carica papaya
gggaaucuugaugaugcugca

SEQ ID NO: 9 - RNA, 21 nt, Solanum lycopersicum
agaaucuugaugaugcugcau

SEQ ID NO: 10 - RNA, 112 nt, Malus domestica
ggucacuuuuugcggguggagcaucaucaygauucacaaucuuacuugguucuaguuuaaccccauuugaugauaugagaaucuugaugaugcugcagcagcaauaaugacu

SEQ ID NO: 11 - RNA, 115 nt, Malus domestica
agucacuuuuugcggguggagcaucuucaagauucacaaucuuauuugggcuccaauuuuagccccauuugaugauaugagaaucuugaugaugcugcagcggcaauaaaugacu

SEQ ID NO: 12 - RNA, 115 nt, Malus domestica
ggucacuuuuugcggguggagcaucaucaagauucacaaucuuacuuggguucuaguuuuaaccccauuugaugauaugagaaucuugaugaugcugcagcagcaauaaaugacu

SEQ ID NO: 13 - RNA, 133 nt, Malus domestica
agucauuguuugcgggugcagcaucaucaagauucacaagugaguagugugacaugauuuaaucgauuuagucaugucgauugaacucgaggaguugagaaucuugaugaugcugcaucagcaauagcugacu

SEQ ID NO: 14 - RNA, 132 nt, Malus domestica
agucauuguuugcgggugccgcaucaccaagauucuuaagugaguagugugacaugauuuaauugauuuaguuaugccgaucggcucacggaguugagaaucuugaugaugcugcaucagcgauggaugacu

SEQ ID NO: 15 - RNA, 133 nt, Malus domestica; identical to SEQ ID NO: 13

SEQ ID NO: 16 - RNA, 105 nt, Malus domestica
gucguuguuugcggguguggcaucaucaagauucacacaugcaauuaacugauaaaguuguuugaaagugagaaucuugaugaugcugcaucggcaauaaaccac

SEQ ID NO: 17 - RNA, 105 nt, Malus domestica; identical to SEQ ID NO: 16

SEQ ID NO: 18 - RNA, 181 nt, Malus domestica
agucaguauucgcgggugcagcaucaucaagauucacauacaggcaagggggcuaccuuuaucgaucgaguaaauuguuaccgcaccaauaagaauauuuccuucaacuuccuuuuguacuugaaagguaguuccuucgaagugggaaucuugaugaugcugcaucagcugguacaugacu

SEQ ID NO: 19 - RNA, 181 nt, Malus domestica; identical to SEQ ID NO: 18

SEQ ID NO: 20 - RNA, 181 nt, Malus domestica; identical to SEQ ID NO: 18

SEQ ID NO: 21 - RNA, 182 nt, Malus domestica
agucaguauucgcgggugcagcaucaucaagauucacauaccuuagcaagggggcuaccuuuagcgaucgaguaaauugguaccacaccaauaugaauauuccuucaacuuccuuuuguacuugaaagguaguuycuucgaagugggaaucuugaugaugcugcagcagcugguacaugacu

SEQ ID NO: 22 - RNA, 112 nt, Malus domestica; identical to SEQ ID NO: 10

SEQ ID NO: 23 - RNA, 115 nt, Malus domestica; identical to SEQ ID NO: 12

SEQ ID NO: 24 - RNA, 115 nt, Malus domestica; identical to SEQ ID NO: 11

SEQ ID NO: 25 - DNA, 105 bp, Malus domestica
gtcgttgtttgtgggcgtggcatcatcaagattcacacatgcaagtaactgctaaagttctttgaaagtgagaatcttgatgatgctgcatctgcaataaaccac

SEQ ID NO: 26 - RNA, 127 nt, Prunus persica
agucguuguuugcgggcguagcaucaucaagauucacgcacaugcaauuaacuugaaaguuucucucuuugccaaaguuucuuucaaagugagaaucuugaugaugcugcaucggcaauaaaccacu

SEQ ID NO: 27 - RNA, 141 nt, Prunus persica
agucauuguuugcgggugcagcaucaccaagauucacaacugaucaagggcacagugacaucaugauguuaugauuuuauuaugacuaugugucuccuaaguuugagaaucuugaugaugcugcaucagcaauagacgacu

SEQ ID NO: 28 - RNA, 168 nt, Prunus persica
ggugcggcaucaucaagauucacauacuuuagugaggggucuaccuuuaucgaucgaguuaauugguacuacuaacaccaccaauugauuuuuguacucgaacuuccuuuaguacucgaaagguaguuccuuugaacuuugaagugggaaucuugaugaugcugcauc

SEQ ID NO: 29 - RNA, 119 nt, Prunus persica
agucauuguuugcggauggagcaucaucaagauucacaauuucuuggggcuagcugcuuugcuauuggcccuuugaugauaugggaaucuugaugaugcugcagcggcaauaaauggcu

SEQ ID NO: 30 - RNA, 162 nt, Citrus sinensis
gucaccuuaaaacagucguugcucgcuguagcagcguccucaagauucacauccagucuaaaggcaaaagcagcaauuuuucuucauuuuugcuugccuugguuuuugucagugagaaucuugaugaugcugcaacggcgauuaaugacuagcuaccaacaa

SEQ ID NO: 31 - RNA, 127 nt, Citrus sinensis
uugcucgcuguagcagcgacgucaagauucacauccagucuaaaggcaaaagcagcaauuuuucuucaguuuggcuugccuggguuuuugucagugagaaucuugaugaugcggcaacggcgaugaa

SEQ ID NO: 32 - RNA, 163 nt, Citrus sinensis
acuguuugcaguuggagcaccaucaagauucacaaacuauuaggguuagugaguggagauaaugguggcuauuuuauuuuuuuuggccccuugcuucacuucaaauugcucuuuguuuuggaaucuugaugaugcugcagcagcgauaaguggcuaaauuaua

SEQ ID NO: 33 - RNA, 110 nt, Vitis vinifera
uauugccgaugcagcaucaucaagauucucauccuugaaaaguuuggcagagauaacaucaccaccgugcauuugcaugugaaucuugaugaugcuacaugcgcaaacaa

SEQ ID NO: 34 - RNA, 118 nt, Vitis vinifera
uauugccgaugcagcaucaucaagauucucaaccccaaaacuugaggcagcgaagauggcaucgcugccgcgccgggcuuucgcaugugaaucuugaugaugcuacaccugcaaacaa

SEQ ID NO: 35 - RNA, 138 nt, Vitis vinifera
guuugcggauggagcaucaucaagauucacaaguauugagacucagugcgugguggugauggugacuuuuguggucccuuccuacacuccgauggcucuuugaugugggaaucuugaugaugcugcagcggcaauaaa

SEQ ID NO: 36 - RNA, 109 nt, Vitis vinifera
guuagcugaugcagcaucaucaagauucacacccaauggaagggcagugaugcaaucucugccaaagauuuugagaugagaaucuugaugaugcugcauucgcagugaa

SEQ ID NO: 37 - RNA, 177 nt, Carica papaya
gagcaucaucaagauucacaaaauuaaacacauuagggcuaguagugugggguuugugguggugggugacggugauggccccuucugcucuuuuuuuuuucaguggucccguugcuuauuucacugcaacacccuuggcucucucucuuugauuaugggaaucuugaugaugcugca

SEQ ID NO: 38 - RNA, 107 nt, Carica papaya
cggcaucaucaagauucacaacaaaacuuuugugaugauaagcucuuuuuaaucgaucgacuugaucaaaagcuucucuccgaaaugggaaucuugaugaugcugca

SEQ ID NO: 39 - RNA, 106 nt, Solanum lycopersicum
auguggcauaaucaagauucacgugaaaaguugcaaauugguuauauaauugaugaaauuaauggcuggcuauuugaaacucacgagaaucuugaugaugcugcau

SEQ ID NO: 40 - RNA, 135 nt, Solanum lycopersicum
auguagcaucaucaagauucauacaugaaaauuaagaggcaagguuaaauauguagcuuuaauuugaaaugaaaaauauaauauaucaugaccaugucuauuuauuucaaagugagaaucuugaugaugcugcau

SEQ ID NO: 41 - DNA, 1978 bp, Malus domestica
ctcgaggattggttttattcctccactattttcctttctgaatgtcctcaaatttttctttctaaatatatgtattctgcgtatcattttcttgttagaatttacagtatttgactcaaatgcatggaggaaatgaatagattgacagtcgttgtttgtgggcgtggcatcatcaagattcacacatgcaagtaactgctaaagttctttgaaagtgagaatcttgatgatgctgcatctgcaataaaccactatatacaacagagaggagactccacagtccacagttttacctttttcacttcaatgatttaattggtatgctttctatctactgtttcaggtaagggtattttttttttttaaattattcttcttcttagttttagaaatttttttatttttgttaacagatggagcccatatatatccatcagtagtgttttcaggtgttatatttatggcctcattgacttggcactgctgactgttggtagatctgtgattattcttctgagaaaagcttgactggggtacaagggttacctgagcaagtctgtttatgcatagagagagagagatgagacattgtctatggtactgggatgagaaagatgagtgttgatcaaccttgacgatgctgcaaagctttaaagcattttgaaaaaggtaaatattttcaaaccatttatatatttttaattttcctaatttgtttggttggttttcagatccagccatggactgtaatccaaaaaactgatttctaatcttttccaaaaatagggaatacattccaataacaaagttgtttggtagaatggttactaaaaacaacaatcttaaaaatgctaaaaggagtccatcattagaactcacaatcaatatttttaaagggaattgattatcaaactttgttttgatcatgattatcattaattttacactcctttgcttaattgtctcttaactgtcttttaatttattcaattcagtcgtttagaaaacagagaggagtataaatcactccccctttggaaatacgaagcaaatattgttagatttcaaagttgggttaaggtctcttcaatataaagatgtttgttttcaaacatgaaaatagtttctcaaatttgttactaaacaaattttaaaactttaaaagttctgaatgcaattaaaatcttcaaattgtatcctcttattacttttgaatcgttaaccaaataacaagcttggtttttttcctttctattgctgagaaatagaaatgtgtgaaagaagttttagattgtctatatgtgtgttgttttttttttcccacctgttttgggtggtgggttgatgcaatttgattcaaagatttgttaattgctttttgaacaatatttcaagtggtttcgatctcttgctgttgagtatatcattgaaaattggaatcggattgcttcggtattcaaaatcttggtgggaacttgtctcactttgtcacattctcactgccattcttgtctttgcctttacacttatttgtggaccaaccaaaaagtgagtcgtcctccaacttgaacctttgagatgtaattagaataatactacacttatcacctatttgtatcattatttatatcatctttctaataaaggaagaacccgctaacacatatgggtctcattagaaaaatgatataaataggtagtaagagtagtattttttagggaactttaacgaaaagcacccggtactgttcactttaacgaaaaaccacattttttcactaaaaagtcaatcctggtactattcactttaccctttattttgtccttatcattaaaactcaaaattttcaagcccttttcattagttttccttattttttatgtataattaacctcactcccctctcattcatcactcctctcaaggaaataatattcagtactccaactattttctcacttcttcaataacagtaac

SEQ ID NO: 42 - DNA, 1963 bp, Malus domestica
gctagaactatacagtactcttaccatacatgagactttaagcttcacactcttctgttttttatacgaaatctcaatttcattggttttaaagcaggttatgtatgagtctcaaccggccatgtgctttacgcagttaagtttaaactatccacataggttaggctctaaaggttgtgacacataagtgggtgtgcgaggatgaaaaaaaaaatgtctaacgtcacattagactattaaaaaaatttccataaacttaagggcatatttacttagcataaatgggaagtaaggaaaaggaatcagatcaactaaacactcccctccccccactagtttatttggtccatgatttgaggtatttgatcaaagatttcatatatactaaaaacctagaaggagaagaagaaactggaaaaagaagatgatatagtgaggcacaaagaagaagattccgcaaggcaaatattaaatcctaactaagaaaaaggtttatgaagaaaatgaagaataaaggaagaggcataacctaacttggagatacataggaaataatttgattccaacaattatcctgctagttaaggcaatttttttaagggtgggattagcctcacaatggactagcaataatgtggttcaaattcgtctttagtgagaatcgaacctaaaacctctcacttacaagtgaagatgaatatcattagaccgtagtattaagtggtagcaattttattctttaaacctcaaatagtcaaaactcatcacaaatcttaatcttaaaatcaaggaataatagtcacttagtactacgtatggactggtagtattcctctttactagtaagtgagaggtcttaggttcgattcttgccaaagcaaaatttgaaccacattattactaacacattatgagacttagcccactctttcaccccttagtatatagacaatatggttcggaaaaaaataaagggataatagggcttacttataagtaattatagaggtaaattgggtttgaaagtgggtcacaaaattagattcatatcaagtttctccttttttaataaacacaaaagaactcaaaaatcaaaacgatttcaagcttgattctttggaagcaacctcaacttctttcagttaacatgatttcgtttagcgtaagtactttgagacccttcaattaatgtaatcaaccaaagaaattgacaatgatataaaaagactgataatgagtggtgcattagacgataccctcatctcccaacacccaaaataaagttagggtttttagtgaagatggcgatgtgtaactgcggccccattggctcagtactgtacactcagcatctaccaagcaaaatgcaaaaattgtaaaaacttggagaaaagtctaggctcaccaatgttccttatatgtaactccaacaaaccgtactttcgttttacaatccgtggttcatgagcttcctaactctgataaatctcacgcgcacacacagggggccaaacgcatgggtcttctctatagtgttagatctcaagcatatatgagctctttttctgtttttattctctgctttatcacgaatttcgtattctattccttttctctctaatctcaaggtattagttctaattttctcctccttttcaaccttattttctactttacacactggataccaactggattttcttctgaatatgactatgtctgtggcatctgaaatggatttttctttttatataaaaaaattaattaataaaattgtgtttttgtgggaatggatcgatcctttgtaaaaacctgagctgcatatcaaatctgtgaattatatacaaaatattgtatcagtactcattgttattattaaatagtaccttaaaagaagaatagggttttgttttgtgcttgggttagattcttctcgggtaccactcttcacgttagcatattg

SEQ ID NO: 43 - DNA, 172 bp, Malus domestica
tattttttagggaactttaacgaaaagcacccggtactgttcactttaacgaaaaaccacattttttcactaaaaagtcaatcctggtactattcactttaccctttattttgtccttatcattaaaactcaaaattttcaagcccttttcattagttttccttatttttta

SEQ ID NO: 44 - RNA, 15 nt, Artificial Sequence
  Construct
gaaucuugaugaugc

SEQ ID NO: 45 - DNA, 20 bp, Artificial Sequence
  Primer
gcacagttgctcctctcaga

SEQ ID NO: 46 - DNA, 24 bp, Artificial Sequence
  Primer
gtacgcagtagaaaggccacatga

SEQ ID NO: 47 - DNA, 2550 bp, Malus domestica
ctcgaggattggttttattcctccattattttcctttctgaatgtcctcaaatttttctttctaagtatatgtattctgcgtatcattttcttgttagaatttacagtatttgactcaaatgcatggaggaaatgaatagattgacagtcgttgtttgtgggcgtggcatcatcaagattcacacatgcaagtaactgctaaagttctttgaaagtgagaatcttgatgatgctgcatctgcaataaaccactatatacaacagagaggagactccacagtccacagttttacctttttcacttcaatgatttaattggtatgctttctatctactgtttcaggtaagggtatttttttttttttaaattattcttcttcttagttttagaattttttttatttttgttaacagatggagcccatatatatccatcagtagtgttttcaggtgttatatttatggcctcattgacttggcactgctgactgttggtagatctgtgattattcttctgagaaaagcttgactggggtacaagggttacctgagcaagtctgtttatgcatagagagagagagatgagacattgtctatggtactgggatgagaaagatgagtgttgatcaaccttgacgatgctgcaaagctttaaagcattttgaaaaaggtaaatattttcaaaccatttatatatttttaattttcctaatttgtttggttggttttcagatccagccatggactgtaatccaaaaaactgatttctaatcttttccaaaatagggaatacattccaataacaaagttgtttggtagaatggttactaaaaacaacaatcttaaaaatgctaaaaggagtccatcattagaactcacaatcaatatttttaaagggaattgattatcaaactttgttttgatcatgattatcattaattttacactcctttgcttaattgtctcttaactgtcttttaatttattcaattcagtcgtttagaaaacagagaggagtataaatcactccccctttggaaatacgaagcaaatattgttagatttcaaagttgggttaagatctcttcaatataaagatgtttgttttcaaacatgaaaatagtttctcaaatttgttactaaacaaattttaaaactttaaaagttctgaatgcaattaaaatcttcaaattgtatcctcttattacttttgaatcgttaaccaaataacaagcttggtttttttcctttctattgctgagaaatagaaatgtgtgaaagaagttttagattgtctatatgtgtgttgtttttttttgcccacctgttttgggtggtgggttgatgcaatttgattcaaagatttgttaattgctttttgaacaatatttcaagtggtttcgatctcttgctgttgagtatatcattgaaaattggaatcggattgcttcggtattcaaaatcttggtgggaacttgtctcactttgtcacattctcactgccattcttgtctttgcctttacacttatttgtggaccaaccaaaaagtgagtcgtcctccaacttgaacctttgagatgtaattagaataatactacacttatcacctatttgtatcattatttatatcatctttctaataaaggaagaacccgctaacacatatgtgtctcattagaaaaatgatataaataggtagtaagagtagtattttttagggaactttaacgaaaagcatccggtactgttcactttaacgaaaaaccacatttttacactaaaaagtcaatcctggtactattcactttaccctttattttgtccttatcattaaaactcaaaattttcaagcccttttcattagttttccttattttttatgtataattaacctcactcccctctcattcatcactcctctcaaggaaataatattcagtactccaactattttctcacttcttcaataacagtaacatccggtgcttcacctttgattcacatattattggatctcaattaggctatgcaataagtactataagaacatgaagatatatctttagtcatatgcaagtatcttctcttcgacggcaatacctatttgtaggaattttttattttctgataatgaagaaaagcttatgaatcacctctatcatcaggtacaacatttagggttaattatgcatacatgtcgatatcaatattcatacatgtatcgaataaattcgagtaatccgaacttaagatttccagttgcaatgatatgacaacttgtttataaagcagtgaactataatgctaggaccactgatgtccaattctcactgttgaggtaggagttgatcttcccttaatgccgagatctttaaactattttagacagatcaccaaaccgtaagaattcggaataaaaatgtacttaaaccaaaccaatcttatagctaaatatatttgtgtatgagttagataaaaagtatatatgaagtataactacctcactctggcttatttagagaacttgcatataaaactaatgttaatgc

SEQ ID NO: 48 - DNA, 172 bp, Malus domestica
tattttttagggaactttaacgaaaagcatccggtactgttcactttaacgaaaaaccacatttttacactaaaaagtcaatcctggtactattcactttaccctttattttgtccttatcattaaaactcaaaattttcaagcccttttcattagttttccttatttttta

SEQ ID NO: 49 - DNA, 171 bp, Malus domestica
tattttttagggaactttaacaaaaagctcctgatacagttcactttaatgaaaaaccacatttttacactaaaaagtcaattctagtactattcactttaccctttattttgacattttcgttaaaactcaaagttttcaagcccctttcattagttttccctattttta

SEQ ID NO: 50 - DNA, 173 bp, Malus domestica
ttaacaaaaagtaaactttaacgcaaaactctcggtactgttcactttaatgaaaaatcatatttttacattaaaaagtcaatcttgttactattcactttaccctttattttatccttatcgttaaaattcaaagttttcaaacccttttcattagttttccttaacaaaat

SEQ ID NO: 51 - DNA, 174 bp, Malus domestica
ttaattaaaagggaactttaacgaaaagctttcggtactgttcattttaacaaaaaatcacatttttacactaaaaagttaatcctgatactattcactttaccctttattttgtccttatatttaaaactcaaagttttcaagcctttttcattagtttttcttaattaaaat

SEQ ID NO: 52 - DNA, 170 bp, Malus sylvestris
tttaatttagggaactttaacgaaaatacctggtactgttcaattaaacgaaaaaccacatttttacactaaaaagtcaatcctggtactatcactttaccatttattttgtccttatcattaaaactcaaagttttcaagcccttttcattagttttccttttaattta

SEQ ID NO: 53 - DNA, 173 bp, Malus domestica
ttaaaataatgggaactttaacgaaaagaagccggtactgttcactttaacgaaaaatcatatttttacactaaaaagtcaatcatggtactattcactttaccctttattttgtacttatcattaaaactcaaagttttcaagccattttcactagttttccttaaaataat

SEQ ID NO: 54 - RNA, 21 nt, Arabidopsis thaliana
agaaucuugaugaugcugcau

SEQ ID NO: 55 - RNA, 21 nt, Arabidopsis thaliana
agaaucuugaugaugcugcag

SEQ ID NO: 56 - RNA, 21 nt, Arabidopsis thaliana
agaaucuugaugaugcugcag

SEQ ID NO: 57 - RNA, 21 nt, Arabidopsis thaliana
agaaucuugaugaugcugcau

SEQ ID NO: 58 - RNA, 21 nt, Arabidopsis thaliana
ggaaucuugaugaugcugcau

SEQ ID NO: 59 - RNA, 21 nt, Malus domestica
agaaucuugaugaugcugcau

SEQ ID NO: 60 - RNA, 21 nt, Malus domestica
agaaucuugaugaugcugcau

SEQ ID NO: 61 - RNA, 21 nt, Malus domestica
ggaaucuugaugaugcugcau

SEQ ID NO: 62 - RNA, 21 nt, Malus domestica
agaaucuugaugaugcugcau

SEQ ID NO: 63 - RNA, 20 nt, Malus domestica
agaaucuugaugaugcugca

SEQ ID NO: 64 - RNA, 21 nt, Malus domestica
ggaaucuugaugaugcugcau

SEQ ID NO: 65 - RNA, 21 nt, Malus domestica
agaaucuugaugaugcugcau

SEQ ID NO: 66 - RNA, 21 nt, Malus domestica
agaaucuugaugaugcugcag

SEQ ID NO: 67 - RNA, 21 nt, Malus domestica
ggaaucuugaugaugcugcag

SEQ ID NO: 68 - RNA, 21 nt, Malus domestica
agaaucuugaugaugcugcag

SEQ ID NO: 69 - RNA, 20 nt, Malus domestica
agaaucuugaugaugcugca

SEQ ID NO: 70 - RNA, 21 nt, Malus domestica
agaaucuugaugaugcugcag

SEQ ID NO: 71 - RNA, 20 nt, Malus domestica
agaaucuugaugaugcugca

SEQ ID NO: 72 - RNA, 21 nt, Malus domestica
ggaaucuugaugaugcugcau

SEQ ID NO: 73 - RNA, 21 nt, Malus domestica
agaaucuugaugaugcugcau

SEQ ID NO: 74 - RNA, 21 nt, Prunus persica
ggaaucuugaugaugcugcag

SEQ ID NO: 75 - RNA, 21 nt, Prunus persica
agaaucuugaugaugcugcau

SEQ ID NO: 76 - RNA, 21 nt, Prunus persica
agaaucuugaugaugcugcau

SEQ ID NO: 77 - RNA, 21 nt, Prunus persica
ggaaucuugaugaugcugcau

SEQ ID NO: 78 - RNA, 20 nt, Citrus sinensis
agaaucuugaugaugcugca

SEQ ID NO: 79 - RNA, 22 nt, Citrus sinensis
uggaaucuugaugaugcugcag

SEQ ID NO: 80 - RNA, 21 nt, Citrus sinensis
agaaucuugaugaugcggcaa

SEQ ID NO: 81 - RNA, 21 nt, Solanum lycopersicum
agaaucuugaugaugcugcau

SEQ ID NO: 82 - RNA, 21 nt, Solanum lycopersicum
agaaucuugaugaugcugcau

SEQ ID NO: 83 - RNA, 21 nt, Vitis vinifera
ugaaucuugaugaugcuacau

SEQ ID NO: 84 - RNA, 21 nt, Vitis vinifera
ggaaucuugaugaugcugcag

SEQ ID NO: 85 - RNA, 21 nt, Vitis vinifera
ugaaucuugaugaugcuacac

SEQ ID NO: 86 - RNA, 21 nt, Carica papaya
gggaaucuugaugaugcugca

SEQ ID NO: 87 - RNA, 21 nt, Carica papaya
gggaaucuugaugaugcugca

SEQ ID NO: 88 - RNA, 22 nt, Artificial Sequence
  Construct
gagaaucuugaugaugcugcau

(19) United States
(12) Patent Application Publication
(10) Pub. No.: US 2018/0223300 A1
(43) Pub. Date: Aug. 9, 2018

(54) Methods and Materials for Producing Fruit of Altered Size

(71) Applicant: The New Zealand Institute for Plant and Food Research Limited, Auckland (NZ)

(72) Inventors: Andrew Peter GLEAVE, Auckland (NZ); Jia-Long YAO, Auckland (NZ)

(73) Assignee: The New Zealand Institute for Plant and Food Research Limited, Auckland (NZ)

(21) Appl. No.: 15/506,390

(22) PCT Filed: Sep. 3, 2015
(86) PCT No.: PCT/IB2015/056677

§ 371 Date: Feb. 24, 2017

(30) Foreign Application Priority Data
Sep. 10, 2014 (NZ) 630787

(51) Int. Cl. C12N 15/82; C12N 15/113

(52) U.S. Cl. CPC C12N 15/8261; C12N 15/8218; C12N 2310/141; C12N 15/113; C12N 15/8249

(57) ABSTRACT

The invention provides materials and methods for producing fruit of altered size, or plants that produce fruit of altered size, by altering expression of miRNA172 in the plants producing the fruit. The invention provides methods and materials for producing the plants and fruit of altered size by genetic modification (GM) and non-GM means. The invention also provides the plants and fruit of altered size. The altered size can be increased or decreased size.

### TECHNICAL FIELD

The present invention relates to methods and materials for producing fruit of altered size.

### BACKGROUND ART

Fruit size is important agronomic trait. Dramatic changes in fruit size have accompanied the domestication of virtually all fruit-bearing crop species, including tomato, watermelon, apple, banana, grape, berries and a vast assortment of other tropical, subtropical, and temperate species.

Despite its fundamental and applied importance, the molecular genetics underlying this important agronomic trait is still poorly understood, particularly in perennial species.

It would be of significant benefit to have tools available useful for genetically manipulating for, and/or accelerating the breeding of plants with, altered fruit size. It would be beneficial to be able to produce, or select for plants, with either increased or decreased fruit size relative to non-manipulated or non-selected plants.

It is therefore an object of the invention to provide novel methods and compositions for producing fruit of altered size, or at least to provide the public with a useful choice.

### SUMMARY OF THE INVENTION

The applicant's invention relates to methods and materials for altering fruit size by manipulating, or selecting, for altered expression of a microRNA (microRNA172, or miRNA172) in plants. Specifically the applicants have shown that when expression of miRNA172 is decreased, fruit size is increased, and conversely when expression of miRNA172 is increased, fruit size is decreased.

The invention has numerous applications for example in genetically modifying plants for the desired fruit size, and in traditional breeding for developing or selecting plants for the desired fruit size.

### Methods

In the first aspect the invention provides a method for altering the size of a fruit, the method comprising altering expression, or activity, of a microRNA172 (miRNA172) in a plant.

In a further aspect the invention provides a method for producing fruit of altered size, the method comprising altering expression, or activity, of an miRNA172 in a plant.

In a further aspect the invention provides a method for producing a plant with fruit of altered size, the method comprising altering expression, or activity, of an miRNA172 in the plant.

Altering includes either increasing or decreasing the size of the fruit.

A fruit of altered size can therefore mean a larger fruit, or a smaller fruit.

Increasing Expression or Activity of an miRNA172 for Smaller Fruit

In one embodiment the expression, or activity, of the miRNA172 is increased, and the fruit size is decreased.

In one embodiment the expression or activity of the miRNA172 is increased by transforming the plant with a polynucleotide encoding the miRNA172.

In a further embodiment the polynucleotide encoding the miRNA172 is operably linked to a promoter sequence.

In one embodiment the promoter is heterologous with respect to the polynucleotide encoding the miRNA172.

In one embodiment the promoter is a promoter which is not normally operably linked to the polynucleotide encoding the miRNA172 in nature.

Decreasing Expression or Activity of a miRNA172 Gene for Larger Fruit

In a further embodiment the expression, or activity, of the miRNA172 is decreased, and the fruit size is increased.

The expression, or activity, of the miRNA172 may be decreased by any means.

Non-GM Selection Method for Selecting a Plant with Altered Fruit Size

In a further aspect the invention provides a method for identifying a plant with a genotype indicative of producing fruit of altered size, the method comprising testing a plant for at least one of:
a) altered expression of at least one miRNA172,
b) altered expression of at least one miRNA172 gene,
c) presence of a marker associated with altered expression of at least one miRNA172, and
d) presence of a marker associated with altered expression of at least one miRNA172 gene.

In one embodiment presence of any of a) to d) indicates that the plant will produce fruit of altered size.

In one embodiment the altered expression is increased expression, and the fruit of altered size is fruit of decreased size.

In a further embodiment the altered expression is decreased expression, and the fruit of altered size is fruit of increased size.

In a further embodiment the method provides the additional step of cultivating the identified plant.

In a further embodiment the method provides the additional step of breeding from the identified plant.

Methods for Breeding Plants with Fruit of Altered Size

In a further aspect the invention provides a method for producing a plant that produces at least one fruit of altered size, the method comprising crossing one of:
a) a plant of the invention,
b) a plant produced by a method of the invention, and
c) a plant selected by a method of the invention,
   with another plant, wherein the off-spring produced by the crossing is a plant that produces at least one fruit of altered size.

In one embodiment the plant produced has increased expression of at least one miRNA172, and the fruit of altered size is fruit of decreased size.

In a further embodiment the altered expression is decreased expression of at least one miRNA172, and the fruit of altered size is fruit of increased size.

### Products

### Constructs

Construct (for Increasing the Expression of at Least One miRNA172 or miRNA172 Gene in a Plant)

In a further aspect the invention provides a construct for increasing the expression of at least one miRNA172 or miRNA172 gene in a plant.

In one embodiment the construct is contains a promoter sequence operably linked to a sequence encoding the miRNA172.

In one embodiment the promoter is a flower-organ-specific promoter.

In a further embodiment promoter is a fruit specific promoter.

In one embodiment the promoter in the construct is heterologous with respect to the sequence encoding the miRNA172.

In one embodiment the promoter in the construct is not normally associated with the sequence encoding the miRNA172 in nature.

Construct (for Reducing or Eliminating Expression of at Least One miRNA172 or miRNA172 Gene in a Plant)

In a further aspect the invention provides a construct for reducing or eliminating expression of at least one miRNA172 or miRNA172 gene in a plant.

In one embodiment the construct is contains a promoter sequence operably linked to at least part of a miRNA172 gene.

In one embodiment the part of the gene is in an antisense orientation relative to the promoter sequence, and forms part of a hair-pin construct for use in RNAi silencing.

In one embodiment the part of a miRNA172 gene is part of the promoter of an endogenous miRNA172 gene.

Preferably the part of the gene is at least 21 nucleotides in length.

This type of construct is useful for transcriptional gene silencing directed toward the promoter of the miRNA172 gene.

Therefore in one embodiment the construct is useful for transcriptional gene silencing directed toward the promoter of the miRNA172 gene.

In a further embodiment the construct includes a promoter linked to a sequence encoding a mutated target site (target mimic) of miRNA172.

In one embodiment the target mimic, includes at least one, preferably at least 2, more preferably at least 3 mismatches relative to the target endogenous miRNA172.

Preferably the mismatches correspond to positions 11 to 13 of the target endogenous miRNA172.

This type of construct is useful for miRNA target mimicry to reduce activity of the target endogenous miRNA172.

Therefore in one embodiment the construct is an miRNA target mimicry construct.

In a further embodiment the construct is an artificial miRNA-directed anti-miRNA construct.

In a further embodiment the artificial miRNA-directed anti-miRNA construct includes a promoter linked to a precursor artificial miRNA (the stem-loop sequences).

The artificial miRNA can be designed to target a mature miRNA172 in order to silence all miRNA172 family members, or it can be designed to target the stem-loop region of a miRNA172 precursor transcript in order to silence only the individual family member to be targeted.

In one embodiment the promoter is a flower-organ-specific promoter.

In a further embodiment promoter is a fruit specific promoter.

In one embodiment the promoter in the construct is heterologous with respect to the at least part of a miRNA172 gene.

In one embodiment the promoter in the construct is not normally associated with the at least part of a miRNA172 gene.

### Fruit of Altered Size

In a further aspect the invention provides a fruit of altered size produced by a method of the invention.

In one embodiment the fruit is of decreased size.

In a further embodiment the fruit is of increased size.

In a further aspect the invention provides a fruit of altered size wherein the fruit has altered expression of at least one miRNA172.

In one embodiment the fruit comprises a construct of the invention.

In one embodiment the altered expression is increased expression, and the fruit of altered size is fruit of decreased size.

In a further embodiment the altered expression is decreased expression, and the fruit of altered size is fruit of increased size.

Plant that Produces Fruit of Altered Size

In a further aspect the invention provides a plant, which produces at least one fruit of altered size, produced by a method of the invention.

In a further aspect the invention provides a plant, which produces at least one fruit of altered size, wherein the plant has altered expression of at least one miRNA172.

In one embodiment the plant comprises a construct of the invention.

In one embodiment the altered expression is increased expression, and the fruit of altered size is fruit of decreased size.

In a further embodiment the altered expression is decreased expression, and the fruit of altered size is fruit of increased size.

### Plant/Fruit

The plant may be from any species that produces fruit.

Preferred plants include apple, pear, peach, kiwifruit, tomato, strawberry, banana and orange plants.

A preferred apple genus is Malus.

Preferred apple species include: Malus angustifolia, Malus asiatica, Malus baccata, Malus coronaria, Malus doumeri, Malus florentina, Malus floribunda, Malus fusca, Malus halliana, Malus honanensis, Malus hupehensis, Malus ioensis, Malus kansuensis, Malus mandshurica, Malus micromalus, Malus niedzwetzkyana, Malus ombrophilia, Malus orientalis, Malus prattii, Malus prunifolia, Malus pumila, Malus sargentii, Malus sieboldii, Malus sieversii, Malus sylvestris, Malus toringoides, Malus transitoria, Malus trilobata, Malus tschonoskii, Malus×domestica, Malus×domestica×Malus sieversii, Malus×domestica×Pyrus communis, Malus xiaojinensis, and Malus yunnanensis.

A particularly preferred apple species is Malus×domestica.

A preferred pear genus is Pyrus.

Preferred pear species include: Pyrus calleryana, Pyrus caucasica, Pyrus communis, Pyrus elaeagrifolia, Pyrus hybrid cultivar, Pyrus pyrifolia, Pyrus salicifolia, Pyrus ussuriensis and Pyrus×bretschneideri.

A particularly preferred pear species are Pyrus communis and Asian pear Pyrus×bretschneideri.

A preferred peach genus is Prunus.

Preferred peach species include: Prunus africana, Prunus apetala, Prunus arborea, Prunus armeniaca, Prunus avium, Prunus bifrons, Prunus buergeriana, Prunus campanulata, Prunus canescens, Prunus cerasifera, Prunus cerasoides, Prunus cerasus, Prunus ceylanica, Prunus cocomilia, Prunus cornuta, Prunus crassifolia, Prunus davidiana, Prunus domestica, Prunus dulcis, Prunus fruticosa, Prunus geniculata, Prunus glandulosa, Prunus gracilis, Prunus grayana, Prunus incana, Prunus incisa, Prunus jacquemontii, Prunus japonica, Prunus korshinskyi, Prunus kotschyi, Prunus laurocerasus, Prunus laxinervis, Prunus lusitanica, Prunus maackii, Prunus mahaleb, Prunus mandshurica, Prunus maximowiczii, Prunus minutiflora, Prunus mume, Prunus murrayana, Prunus myrtifolia, Prunus nipponica, Prunus occidentalis, Prunus padus, Prunus persica, Prunus pleuradenia, Prunus pseudocerasus, Prunus prostrata, Prunus salicina, Prunus sargentii, Prunus scoparia, Prunus serrula, Prunus serrulate, Prunus sibirica, Prunus simonii, Prunus sogdiana, Prunus speciosa, Prunus spinosa, Prunus spinulosa, Prunus ssiori, Prunus subhirtella, Prunus tenella, Prunus tomentosa, Prunus triloba, Prunus tumeriana, Prunus ursina, Prunus vachuschtii, Prunus verecunda, Prunus xyedoensis, Prunus zippeliana, Prunus alabamensis, Prunus alleghaniensis, Prunus americana, Prunus andersonii, Prunus angustifolia, Prunus brigantina, Prunus buxifolia, Prunus caroliniana, Prunus cuthbertii, Prunus emarginata, Prunus eremophila, Prunus fasciculate, Prunus fremontii, Prunus geniculata, Prunus gentryi, Prunus havardii, Prunus hortulana, Prunus huantensis, Prunus ilicifolia, Prunus integrifolia, Prunus maritima, Prunus mexicana, Prunus munsoniana, Prunus nigra, Prunus pensylvanica, Prunus pumila, Prunus rigida, Prunus rivularis, Prunus serotina, Prunus sphaerocarpa, Prunus subcordata, Prunus texana, Prunus umbellate and Prunus virginiana.

A particularly preferred peach species is Prunus persica.

A preferred kiwifruit genus is Actinidia.

Preferred kiwifruit species include: Actinidia arguta, Actinidia arisanensis, Actinidia callosa, Actinidia carnosifolia, Actinidia chengkouensis, Actinidia chinensis, Actinidia chrysantha, Actinidia cinerascens, Actinidia cordifolia, Actinidia coriacea, Actinidia cylindrica, Actinidia deliciosa, Actinidia eriantha, Actinidia farinosa, Actinidia fasciculoides, Actinidia fortunatii, Actinidia foveolata, Actinidia fulvicoma, Actinidia glauco-callosa-callosa, Actinidia glaucophylla, Actinidia globosa, Actinidia gracilis, Actinidia grandiflora, Actinidia hemsleyana, Actinidia henryi, Actinidia holotricha, Actinidia hubeiensis, Actinidia indochinensis, Actinidia kolomikta, Actinidia laevissima, Actinidia lanceolata, Actinidia latifolia, Actinidia leptophylla, Actinidia liangguangensis, Actinidia lijiangensis, Actinidia linguiensis, Actinidia longicarpa, Actinidia macrosperma, Actinidia maloides, Actinidia melanandra, Actinidia melliana, Actinidia obovata, Actinidia oregonensis, Actinidia persicina, Actinidia pllosula, Actinidia polygama, Actinidia purpurea, Actinidia rongshuiensis, Actinidia rubricaulis, Actinidia rubus, Actinidia rudis, Actinidia rufa, Actinidia rufotricha, Actinidia sabiaefolia, Actinidia sorbifolia, Actinidia stellato-pllosa-pllosa, Actinidia styracifolia, Actinidia suberifolia, Actinidia tetramera, Actinidia trichogyna, Actinidia ulmifolia, Actinidia umbelloides, Actinidia valvata, Actinidia venosa, Actinidia vitifolia and Actinidia zhejiangensis.

Particularly preferred kiwifruit species are Actinidia arguta, Actinidia chinensis and Actinidia deliciosa.

A preferred tomato genus is Solanum.

A preferred tomato species is Solanum lycopersicum.

A preferred banana genus is Musa.

Preferred banana species include: Musa acuminata, Musa balbisiana, and Musa×paradisiaca

A preferred orange genus is Citrus.

Preferred orange species include: Citrus aurantiifolia, Citrus crenatifolia, Citrus maxima, Citrus medica, Citrus reticulata, Citrus trifoliata, Australian limes Citrus australasica, Citrus australis, Citrus glauca, Citrus garrawayae, Citrus gracilis, Citrus inodora, Citrus warburgiana, Citrus wintersii, Citrus japonica, Citrus indica and Citrus×sinensis.

Particularly preferred orange species are: Citrus maxima, Citrus reticulate, Citrus×sinensis

A preferred grape genus is Vitis.

Preferred grape species include: Vitis vinifera, Vitis labrusca, Vitis riparia, Vitis aestivalis, Vitis rotundifolia, Vitis rupestris, Vitis coignetiae, Vitis amurensis, Vitis vulpine.

A particularly preferred grape species is Vitis vinifera.

In a preferred embodiment the plant is from a species that produces accessory fruit.

### Accessory Fruit

Unlike true fruit which are derived from ovary tissue, accessory fruits are derived from other floral or receptacle tissue.

Fruit Derived from Hypanthium Tissue

Preferred accessory fruit species include those in which the fruit flesh is derived from hypanthium tissue. The hypanthium is a tube of sepal, petal and stamen tissue surrounding the carpel.

Preferred plants for which fruit flesh is derived from hypanthium tissue include apple and pear plants (as described above). Other preferred plants in which the fruit flesh is derived from hypanthium tissue include quince, loquat, and hawthorn.

A preferred quince genus is Chaenomeles. Preferred quince species include: Chaenomeles cathayensis and Chaenomeles speciosa. A particularly preferred quince species is Chaenomeles speciosa.

A preferred loquat genus is Eriobotrya. Preferred loquat species include: Eriobotrya japonica and Eriobotrya japonica. A particularly preferred loquat species is Eriobotrya japonica

A preferred hawthorn genus is Crataegus. Preferred hawthorn species include: Crataegus azarolus, Crataegus columbiana, Crataegus crus-galli, Crataegus curvisepala, Crataegus laevigata, Crataegus mollis, Crataegus monogyna, Crataegus nigra, Crataegus rivularis, and Crataegus sinaic.

### Plant Parts, Propagules and Progeny

In a further embodiment the invention provides a part, progeny, or propagule of a plant of the invention.

Preferably the part, progeny, or propagule has altered expression of at least one miRNA172 or miRNA172 gene.

Preferably the part, progeny, propagule comprises a construct of the invention.

The term “part” of a plant refers to any part of the plant. The term “part” preferably includes any one of the following: tissue, organ, fruit, and seed.

The term “propagule” of a plant preferably includes any part of a plant that can be used to regenerate a new plant. Preferably the term “propagule” includes seeds and cuttings.

The term “progeny” includes any subsequent generation of plant. The progeny may be produced as a result of sexual crossing with another plant. The progeny plant may also be asexually produced.

### DETAILED DESCRIPTION OF THE INVENTION

### Definitions

### Fruit Size

The term fruit size refers to the volume of the fruit.

A convenient way to assess the volume of the fruit may be to measure the diameter of the fruit, or the weight of the fruit.

### Altered Fruit Size

The term altered fruit size means that the fruit are altered in size relative to those of a control plant.

The altered fruit size may be either increased or decreased fruit size. In one embodiment the altered fruit size is increased fruit size. In a further embodiment the altered fruit size is decreased fruit size.

The control plant may be at least one of:
a wild type plant
a non-transformed plant
a plant transformed with a control construct
a non selected plant

### MicroRNAs

MicroRNAs (abbreviated miRNAs) are small RNA molecules with a length of 20-22 nt (nucleotide), present in eukaryotes and encoded by the genomes of the eukaryotes. miRNAs recognize target genes mainly by complementarily pairing with the RNA of target genes and then inhibit the expression of the target genes through miRNA-RISC (RNA induced silence complex) (Jones-Rhoades M W, Bartel D P, and Bartel B. MicroRNAs and their regulatory roles in plants. Annual Review of Plant Biology, 2006, 57: 19-53).

Each miRNA gene produces at least three RNA species, including:
a pri-miRNA,
a pre-miRNA, and
the mature miRNA

These are produced through sequential endonucleolytic maturation steps (Kim V N MicroRNA biogenesis: coordinated cropping and dicing. Nat Rev Mol Cell Biol 2005, 6: 376-385).

The pri-miRNA is the primary transcript ranges in size from about 60 to about 2000 nucleotides in length. pri-miRNA are structurally similar to standard messenger RNAs (mRNAs), having such features as 5′-CAP and 3′ poly(A). Therefore pri-miRNAs can be cloned into, or identified in conventional cDNA libraries.

The intermediate pre-miRNAs (precursor miRNAs) are about 60 nucleotides in length. Pre-miRNAs form a stable foldback secondary structure that is recognized by an enzyme necessary for miRNA maturation.

Processing of the pre-miRNA results in production of the mature miRNA of about 20-22 nt (nucleotide) nucleotides in length.

While pre-miRNA molecules may have several very small ORFs, no pre-miRNA molecules from which a protein can be translated have been found.

Pre-miRNAs from which miRNAs are formed are located in the transcripts of miRNA genes, and are usually of 60 nt to 200 nt in length.

miRNAs have important regulatory roles during plant development, growth, and in response to biological and non-biological stresses. The target genes of many miRNAs belong to transcription factor family. The same miRNA may often inhibit the functions of a variety of target genes, while regulating various interconnected processes during plant development and growth.

For example, overexpression of miRNA156 increases the number of leaves of Arabidopsis thaliana more than 100 times and plant dry weight 5 times, and delays flowering time (Wu G and Poethig R S. Temporal regulation of shoot development in Arabidopsis thaliana by miR156 and its target SPL3. Development, 2006, 133: 3539-3547).

In corn, miRNA172 regulates the sex differentiation of flower organ in addition to flowering time (Chuck G, Meeley R, Irish E, Sakai H, and Hake S. The maize tasselseed4 microRNA controls sex determination and meristem cell fate by targeting Tasselseed6/indeterminate spikelet1. Nat Genet, 2007, 39: 1517-1521).

miRNA172

Like other miRNAs, miRNA172 has been shown to regulate various processes in plants. In maize microRNA172 has been reported to down-regulate glossy15 to and thereby promote vegetative phase change (Lauter et al., Proc Natl Acad Sci USA. 2005 Jun. 28; 102(26):9412-7. Epub 2005 Jun. 15.) In barley interaction between alleles of HvAPETALA2 and microRNA172 has been reported to determine the density of grains on the inflorescence. In Arabidopsis interaction between miRNA172, Gigantea (GI), and WRKY44 has been proposed to regulate drought escape and drought tolerance by affecting sugar signalling (Han et al, PLoS One. 2013 Nov. 6; 8(11):e73541. doi: 10.1371/journal.pone.0073541. eCollection 2013.).

miRNA172 sequences, and the genes encoding them, are well known in the art.

miRNA172 is found in many plant species and is highly conserved.

In one embodiment the miRNA172 is 21 nucleotides in length.

In one embodiment the miRNA172 comprises a sequence with at least 70% identity to any one of the miRNA172 sequences referred to in Table 1 below, and shown in the sequence listing.

In a further embodiment the miRNA172 comprises the consensus sequence of SEQ ID NO: 1.

In a further embodiment the miRNA172 comprises the conserved sequence of SEQ ID NO: 44.

In a further embodiment the miRNA172 comprises a sequence with at least 70% identity to the sequence of SEQ ID NO:2.

In a further embodiment the miRNA172 comprises a sequence a miRNA172 sequences referred to in Table 1 below, and shown in the sequence listing.

In a further embodiment the miRNA172 comprises the sequence of SEQ ID NO:2.

### MicroRNA172 Genes

In one embodiment the miRNA172 gene encodes an miRNA172 as defined above.

In a further embodiment the miRNA172 gene comprises a sequence with at least 70% identity to any one of the miRNA172 gene sequences referred to in Table 1 below, and shown in the sequence listing.

In a further embodiment the miRNA172 gene comprises a sequence with at least 70% identity to the sequence of SEQ ID NO:41.

In a further embodiment the miRNA172 gene comprises a sequence of any one of the miRNA172 gene sequences referred to in Table 1 below, and shown in the sequence listing.

In a further embodiment the miRNA172 gene comprises the sequence of SEQ ID NO:41.

TABLE 1
miRNA172 sequences

| SEQ | | | | |
|---|---|---|---|---|
| ID | Sequence | Common | | |
| NO: | type | name | Species | Reference |
| 1 | miRNA172 | N/A | N/A | Consensus sequence |
| 2 | miRNA172 | Apple | Malus × | Mdm-miRNA172p |
| | | | domestica | |
| 3 | miRNA172 | Pear | Pyrus | Pbr-miRNA172p |
| | | | bretschneideri | |
| 4 | miRNA172 | Pear | Pyrus | Pco-miRNA172p |
| | | | communis | |
| 5 | miRNA172 | peach | Prunus persica | Ppe-miR172a |
| 6 | miRNA172 | orange | Citrus × | Csi-miRNA172a |
| | | | sinensis | |
| 7 | miRNA172 | grape | Vitis vinifera | Vvi-miRNA172a |
| 8 | miRNA172 | papaya | Carica papaya | Cpa-miR172a |
| 9 | miRNA172 | tomato | Solanum | Sly-miR172a |
| | | | lycopersicum | |
| 10 | miRNA172 | Apple | Malus × | Mdm-miRNA172a |
| | precursor | | domestica | |
| 11 | miRNA172 | Apple | Pyrus | Mdm-miRNA172b |
| | precursor | | bretschneideri | |
| 12 | miRNA172 | Apple | Pyrus | Mdm-miRNA172c |
| | precursor | | communis | |
| 13 | miRNA172 | Apple | Malus × | Mdm-miRNA172d |
| | precursor | | domestica | |
| 14 | miRNA172 | Apple | Malus × | Mdm-miRNA172e |
| | precursor | | domestica | |
| 15 | miRNA172 | Apple | Malus × | Mdm-miRNA172f |
| | precursor | | domestica | |
| 16 | miRNA172 | Apple | Malus × | Mdm-miRNA172g |
| | precursor | | domestica | |
| 17 | miRNA172 | Apple | Malus × | Mdm-miRNA172h |
| | precursor | | domestica | |
| 18 | miRNA172 | Apple | Malus × | Mdm-miRNA172i |
| | precursor | | domestica | |
| 19 | miRNA172 | Apple | Malus × | Mdm-miRNA172j |
| | precursor | | domestica | |
| 20 | miRNA172 | Apple | Malus × | Mdm-miRNA172k |
| | precursor | | domestica | |
| 21 | miRNA172 | Apple | Malus × | Mdm-miRNA172l |
| | precursor | | domestica | |
| 22 | miRNA172 | Apple | Malus × | Mdm-miRNA172m |
| | precursor | | domestica | |
| 23 | miRNA172 | Apple | Malus × | Mdm-miRNA172n |
| | precursor | | domestica | |
| 24 | miRNA172 | Apple | Malus × | Mdm-miRNA172o |
| | precursor | | domestica | |
| 25 | miRNA172 | Apple | Malus × | Mdm-miRNA172p |
| | precursor | | domestica | |
| 26 | miRNA172 | Peach | Prunus persica | Ppe-miRNA172a |
| | precursor | | | |
| 27 | miRNA172 | Peach | Prunus persica | Ppe-miRNA172b |
| | precursor | | | |
| 28 | miRNA172 | Peach | Prunus persica | Ppe-miRNA172c |
| | precursor | | | |
| 29 | miRNA172 | Peach | Prunus persica | Ppe-miRNA172d |
| | precursor | | | |
| 30 | miRNA172 | Orange | Citrus sinensis | Csi-miRNA172a |
| | precursor | | | |
| 31 | miRNA172 | Orange | Citrus sinensis | Csi-miRNA172b |
| | precursor | | | |
| 32 | miRNA172 | Orange | Citrus sinensis | Csi-miRNA172c |
| | precursor | | | |
| 33 | miRNA172 | Grape | Vitis vinifera | Csi-miRNA172a |
| | precursor | | | |
| 34 | miRNA172 | Grape | Vitis vinifera | Csi-miRNA172b |
| | precursor | | | |
| 35 | miRNA172 | Grape | Vitis vinifera | Csi-miRNA172c |
| | precursor | | | |
| 36 | miRNA172 | Grape | Vitis vinifera | Csi-miRNA172d |
| | precursor | | | |
| 37 | miRNA172 | Papaya | Carica papaya | Cpa-miRNA172a |
| | precursor | | | |
| 38 | miRNA172 | Papaya | Carica papaya | Cpa-miRNA172b |
| | precursor | | | |
| 39 | miRNA172 | Tomato | Solanum | Sly-miRNA172a |
| | precursor | | lycopersicum | |
| 40 | miRNA172 | Tomato | Solanum | Sly-miRNA172b |
| | precursor | | lycopersicum | |
| 41 | miRNA172 | Apple | Malus × | Mdm-miRNA172p |
| | gene | | domestica | |
| 42 | miRNA172 | Apple | Malus × | Mdm-miRNA172p |
| | promoter | | domestica | |
| 43 | Transposable | Apple | Malus × | Mdm-miRNA172p |
| | element | | domestica | |
| 44 | miRNA172 | N/A | N/A | Completely conserved |
| | | | | region |

A cloned miRNA172 sequence may of course be used as a probe or primer to identify further miRNA172, miRNA172 genes and promoters from other species, using methods well known to those skilled in the art and described herein.

### Gene

A term “gene” as used herein may be the target for reducing, or eliminating, expression of a miRNA172 or miRNA172 gene.

The term gene include the sequence encoding the protein, which may be separate exons, any regulatory sequences (including promoter and terminator sequences) 5′ and 3′ untranslated sequence, and introns.

It is known by those skilled in the art that any of such features of the gene may be targeted in silencing approaches such as antisense, sense suppression and RNA interference (RNAi).

Altered microRNA Activity

The terms reduced expression, reducing expression and grammatical equivalents thereof mean reduced/reducing expression relative to that in at least one of:
a wild type plant
a non-transformed plant
a plant transformed with a control construct
a non selected plant

A control construct may be for example an empty vector construct.

Methods for Increasing the Expression of miRNA172

Methods for increasing the expression of miRNA172 will be readily apparent to those skilled in the art. For example a sequence encoding an miRNA172, such as a pri-miRNA172 can be cloned operably linked a suitable promoter, to drive expression of the pri-miRNA172, leading to function processing to produce the mature miRNA172 in the plant.

Such cloning and expression methods are well-known to those skilled in the art and are described herein and demonstrated in the Examples.

Methods for Repressing microRNA Activity

Methods for repressing microRNA activity are also well-known to those skilled in the art and are described for example in Eamens and Wang (Plant Signaling & Behaviour 6:3, 349-359, 2001).

Methods for repressing the activity of miRNA172 according to the invention include but are not limited to transcriptional gene silencing, miRNA target mimicry, and artificial miRNA-directed anti-miRNA technology, all of which are described in Eamens and Wang (Plant Signaling & Behaviour 6:3, 349-359, 2011).

The expression, or activity, of the miRNA172 may thus be decreased by any means.

### Transcriptional Gene Silencing

In one embodiment the expression, or activity, of the miRNA172 is decreased by transcriptional gene silencing.

In one embodiment the expression of an endogenous gene encoding the miRNA172 is suppressed.

In one embodiment the endogenous gene is suppressed by RNAi silencing.

In a further embodiment the RNAi silencing is affected by introducing an RNAi construct targeting the endogenous gene.

In one embodiment the RNAi construct targets the promoter of the endogenous gene.

This approach is useful for silencing individual members of a family of miRNA172 sequences in species where such families are found.

miRNA Target Mimicry

In a further embodiment, expression, or activity, of the miRNA172 is decreased by miRNA target mimicry.

This approach is useful for silencing multiple members of a family of miRNA172 sequences in species where such families are found.

Artificial miRNA-Directed Anti-miRNA Technology

In a further embodiment, expression, or activity, of the miRNA172 is decreased by artificial miRNA-Directed Anti-miRNA technology

### Targetted Expression of Expression or Silencing Constructs

When expressing sequences in the approaches discussed above, it may be useful to use a tissue- or developmental stage-specific promoter. This may for example be useful for targeting a particular tissue or developmental stage to express the miRNA172. Alternatively this approach may be useful to target the silencing of only an miRNA172, or miRNA172, expressed in a particular tissue or at a particular developmental stage.

### Tissue Specific Promoters

Tissue specific promoters are known to those skilled in the art.

Suitable tissue specific promoters include flower-organ-specific promoters, and fruit-specific promoters.

Suitable flower-organ-specific promoters include, but are not limited to; ovary-specific promoters, such as the TPRP-F1 promoter for the tomato proline-rich protein gene (Carmi et al., Induction of parthenocarpy in tomato via specific expression of the rolB gene in the ovary. Planta, 2003. 217(5): p. 726-735.), for altering miRNA172 expression or activity to regulate the size of fruit developed from ovary tissues; and sepal-specific promoters, such as the promoter of MdMADS5/MdAP1. (Mimida et. al., Expression patterns of several floral genes during flower initiation in the apical buds of apple (Malus×domestica Borkh.) revealed by in situ hybridization. Plant Cell Reports, 2011. 30(8): p. 1485-1492.) gene, for altering miRNA172 expression or activity to regulate the size of fruit developed from hypanthium tissues.

Suitable fruit-specific promoters include, but are not limited to; the promoters of the MdMADS6, 7, 8 and 9 genes (Yao et al., Seven MADS-box genes in apple are expressed in different parts of the fruit. Journal of the American Society for Horticultural Science, 1999. 124(1): p. 8-13.) that drive gene expression from early stages of fruit development and response to pollination induced gene expression.

Methods for Detecting Altered Expression of miRNA172

Methods for detecting altered expression of miRNA172 are well known to those skilled in the art. For example, quantitative RT-PCR analyses (Drummond, R. S. M. et al. Plant Physiology 151, 1867-1877, 2009) may be used for determine the relative levels of miRNA precursor. In addition, the stem-loop RT-PCR miRNA assay (Varkonyi-Gasic, E., Wu, R., Wood, M., Walton, E. F. & Hellens, R. P. Protocol: a highly sensitive RT-PCR method for detection and quantification of microRNAs. Plant Methods 3, 2007), may be used for determine the relative levels of mature miRNA.

### Marker Assisted Selection

Marker assisted selection (MAS) is an approach that is often used to identify plants that possess a particular trait using a genetic marker, or markers, associated with that trait. MAS may allow breeders to identify and select plants at a young age and is particularly valuable for fruit traits that are hard to measure at a young stage. The best markers for MAS are the causal mutations, but where these are not available, a marker that is in strong linkage disequilibrium with the causal mutation can also be used. Such information can be used to accelerate genetic gain, or reduce trait measurement costs, and thereby has utility in commercial breeding programs.

Methods for marker assisted selection are well known to those skilled in the art, for example: (Collard, B. C. Y. and D. J. Mackill, Marker-assisted selection: an approach for precision plant breeding in the twenty-first century. Philosophical Transactions of the Royal Society B—Biological Sciences, 2008. 363(1491): p. 557-572.)

### Markers

Markers for use in the methods of the invention may include nucleic acid markers, such as single nucleotide polymorphisms (SNPs), simple sequence repeats (SSRs or microsatellites), insertions, substitutions, indels and deletions.

Preferably the marker is in linkage disequilibrium (LD) with the trait.

Preferably the marker is in LD with the trait at a D′ value of at least 0.1, more preferably at least 0.2, more preferably at least 0.3, more preferably at least 0.4, more preferably at least 0.5.

Preferably the marker is in LD with the trait at a R² value of at least 0.05, more preferably at least 0.075, more preferably at least 0.1, more preferably at least 0.2, more preferably at least 0.3, more preferably at least 0.4, more preferably at least 0.5.

The term “linkage disequilibrium” or LD as used herein, refers to a derived statistical measure of the strength of the association or co-occurrence of two independent genetic markers. Various statistical methods can be used to summarize linkage disequilibrium (LD) between two markers but in practice only two, termed D′ and R², are widely used.

Markers linked, and or in LD, with the trait may be of any type including but not limited to, SNPs, substitutions, insertions, deletions, indels, simple sequence repeats (SSRs).

In the present invention, markers are associated with altered expression of miRNA172.

One such marker identified by the applicant is the presence of a transposable element (TE). The sequence of the TE is shown in SEQ ID NO:43.

To genotype the miRNA172p locus, PCR amplification can be performed using primers located up-stream and down-stream of the TE insertion. The amplification results in a small fragment from the CAFS allele of miRNA172p containing no TE insertion, and results in a large fragment from the cafs allele containing the TE. The cafs allele (including the TE) reduces miRNA172 expression and increases fruit size, while the CAFS allele (without the TE) decreases fruit size. This is further explained in Example 1. Suitable primer sequences for the primers and TE are shown in FIG. 6.

Therefore in one embodiment the marker comprises the sequence shown in SEQ ID NO:43.

Other Markers Linked to miRNA172.

It would be most desirable to identity the presence of the TE discussed above when selecting for large fruit. However, following the applicants present disclosure, those skilled in the art would know that it would also be possible to select for large fruit by identifying the presence of a marker linked to the TE. Selection methods utilising such linked markers also form part of the present invention. Methods for identify such linked markers are known to those skilled in the art, and are shown in the present Examples. Furthermore, by way of example, several markers linked to the TE are shown in FIG. 2b.

Therefore in a further embodiment the marker comprises any one of the markers shown in FIG. 2b.

### Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

Preferably the term “polynucleotide” includes both the specified sequence and its compliment.

A “fragment” of a polynucleotide sequence provided herein is a subsequence of contiguous nucleotides, e.g., a sequence that is at least 15 nucleotides in length. The fragments of the invention comprise 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide of the invention.

Fragments of polynucleotides for use in silence, in particular for RNA interference (RNAi) approaches are preferably at least 21 nucleotides in length.

The term “primer” refers to a short polynucleotide, usually having a free 3′OH group that is hybridized to a template and used for priming polymerization of a polynucleotide complementary to the target.

The term “isolated” as applied to the polynucleotide sequences disclosed herein is used to refer to sequences that are removed from their natural cellular environment. In one embodiment the sequence is separated from its flanking sequences as found in nature. An isolated molecule may be obtained by any method or combination of methods including biochemical, recombinant, and synthetic techniques.

The term “recombinant” refers to a polynucleotide sequence that is synthetically produced or is removed from sequences that surround it in its natural context. The recombinant sequence may be recombined with sequences that are not present in its natural context.

The term “derived from” with respect to polynucleotides being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

### Variants

As used herein, the term “variant” refers to polynucleotide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the polynucleotides disclosed herein possess biological activities that are the same or similar to those of the disclosed polynucleotides. The term “variant” with reference to polypeptides and polynucleotides encompasses all forms of polypeptides and polynucleotides as defined herein.

### Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present invention. Identity is found over a comparison window of at least 20 nucleotide positions, preferably at least 50 nucleotide positions, more preferably at least 100 nucleotide positions, and most preferably over the entire length of a polynucleotide of the invention.

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). In one embodiment the default parameters of bl2seq are utilized. In a further except the default parameters of bl2seq are utilized, except that filtering of low complexity parts should be turned off.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

A preferred method for calculating polynucleotide % sequence identity is based on aligning sequences to be compared using Clustal X (Jeanmougin et al., 1998, Trends Biochem. Sci. 23, 403-5.)

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp://ftp.ncbi.nih.gov/blast/).

Alternatively, variant polynucleotides of the present invention hybridize to the specified polynucleotide sequences, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency. With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C-log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)o C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

### Constructs, Vectors and Components Thereof

The term “genetic construct” refers to a polynucleotide molecule, usually double-stranded DNA, which may have inserted into it another polynucleotide molecule (the insert polynucleotide molecule) such as, but not limited to, a cDNA molecule or an miRNA encoding molecule. A genetic construct may contain the necessary elements that permit transcribing the insert polynucleotide molecule. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

In one embodiment at least one of the promoter and terminator is heterologous with respect to the polynucleotide to be expressed. In one embodiment the promoter is heterologous with respect to the polynucleotide to be expressed. In a further embodiment the terminator is heterologous with respect to the polynucleotide to be expressed. The term “heterologous” means that the sequences, that are heterologous to each other, are not found together in nature. Preferably the sequences are not found operably linked in nature. In one embodiment, the heterologous sequences are found in different species. However, one or more of the heterologous sequences may also be synthetically produced and not found in nature at all.

“Operably-linked” means that the sequence of interest, such as a sequence to be expressed is placed under the control of, and typically connected to another sequence comprising regulatory elements that may include promoters, tissue-specific regulatory elements, temporal regulatory elements, enhancers, repressors and terminators, 5′-UTR sequences, 5′-UTR sequences comprising uORFs, and uORFs.

The term “noncoding region” refers to untranslated sequences that are upstream of the translational start site and downstream of the translational stop site. These sequences are also referred to respectively as the 5′-UTR and the 3′-UTR. These regions include elements required for transcription initiation and termination and for regulation of translation efficiency.

A 5′-UTR sequence is the sequence between the transcription initiation site, and the translation start site.

The 5′-UTR sequence is an mRNA sequence encoded by the genomic DNA. However as used herein the term 5′-UTR sequence includes the genomic sequence encoding the 5′-UTR sequence, and the compliment of that genomic sequence, and the 5′-UTR mRNA sequence.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to cis-regulatory elements upstream of the coding region that regulate gene transcription. Promoters comprise cis-initiator elements which specify the transcription initiation site and conserved boxes such as the TATA box, and motifs that are bound by transcription factors.

A “transgene” is a polynucleotide that is introduced into an organism by transformation. The transgene may be derived from the same species or from a different species as the species of the organism into which the transgene is introduced. The transgenet may also be synthetic and not found in nature in any species.

A “transgenic plant” refers to a plant which contains new genetic material as a result of genetic manipulation or transformation. The new genetic material may be derived from a plant of the same species as the resulting transgenic plant or from a different species, or may be synthetic.

Preferably the “transgenic” is different from any plant found in nature due the presence of the transgene.

An “inverted repeat” is a sequence that is repeated, where the second half of the repeat is in the complementary strand, e.g.,

(5′)GATCTA . . . TAGATC(3′)
(3′)CTAGAT . . . ATCTAG(5′)

Read-through transcription will produce a transcript that undergoes complementary base-pairing to form a hairpin structure provided that there is a 3-5 bp spacer between the repeated regions.

The terms “to alter expression of” and “altered expression” of a polynucleotide of the invention, are intended to encompass the situation where genomic DNA corresponding to a polynucleotide of the invention is modified thus leading to altered expression of a polynucleotide or polypeptide of the invention. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

### Methods for Isolating or Producing Polynucleotides

The polynucleotide molecules of the invention can be isolated by using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser, incorporated herein by reference. The polynucleotides of the, or for use in methods of the invention can be amplified using primers, as defined herein, derived from the polynucleotide sequences of the invention.

Further methods for isolating polynucleotides include use of all, or portions of, the polypeptides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments may be produced by techniques well-known in the art such as restriction endonuclease digestion, oligonucleotide synthesis and PCR amplification.

A partial polynucleotide sequence may be used, in methods well-known in the art to identify the corresponding full length polynucleotide sequence. Such methods include PCR-based methods, 5′RACE (Frohman M A, 1993, Methods Enzymol. 218: 340-56) and hybridization-based method, computer/database—based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilise a sequence identical (or at least highly similar) to that in the plant, for which reduced expression is desired. For these reasons among others, it is desirable to be able to identify and isolate orthologues of a particular gene in several different plant species.

Variants (including orthologues) may be identified by the methods described.

### Methods for Identifying Variants

### Physical Methods

Variant polypeptides may be identified using PCR-based methods (Mullis et al., Eds. 1994 The Polymerase Chain Reaction, Birkhauser). Typically, the polynucleotide sequence of a primer, useful to amplify variants of polynucleotide molecules of the invention by PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides of the invention (Sambrook et al., Molecular Cloning: A Laboratory Manual, 2nd Ed. Cold Spring Harbor Press, 1987) or by identifying polypeptides from natural sources with the aid of such antibodies.

### Computer Based Methods

The variant sequences of the invention, including both polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

### Methods for Modifying Sequences

Methods for modifying the sequence of proteins, or the polynucleotide sequences encoding them, are well known to those skilled in the art. The sequence of a protein may be conveniently be modified by altering/modifying the sequence encoding the protein and expressing the modified protein. Approaches such as site-directed mutagenesis may be applied to modify existing polynucleotide sequences. Alternatively restriction endonucleases may be used to excise parts of existing sequences. Altered polynucleotide sequences may also be conveniently synthesised in a modified form.

### Methods for Producing Constructs and Vectors

The genetic constructs of the present invention comprise one or more polynucleotide sequences of the invention and/or polynucleotides encoding polypeptides of the invention, and may be useful for transforming, for example, bacterial, fungal, insect, mammalian or plant organisms. The genetic constructs of the invention are intended to include expression constructs as herein defined. Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

### Methods for Producing Host Cells Comprising Polynucleotides, Constructs or Vectors

The invention provides a host cell which comprises a genetic construct or vector of the invention. Host cells may be derived from, for example, bacterial, fungal, insect, mammalian or plant organisms.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

### Methods for Producing Plant Cells and Plants Comprising Constructs and Vectors

The invention further provides plant cells which comprise a genetic construct of the invention, and plant cells modified to alter expression of a polynucleotide or polypeptide of the invention. Plants comprising such cells also form an aspect of the invention.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

### Methods for Genetic Manipulation of Plants

A number of plant transformation strategies are available (e.g. Birch, 1997, Ann Rev Plant Phys Plant Mol Biol, 48, 297, Hellens R P, et al (2000) Plant Mol Biol 42: 819-32, Hellens R et al (2005) Plant Meth 1: 13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species. Transformation strategies may be designed to reduce, or eliminate, expression of a polynucleotide/polypeptide in a plant cell, tissue, organ or at a particular developmental stage which/when it is normally expressed. Such strategies are known as gene silencing strategies.

Genetic constructs for expression of genes in transgenic plants typically include promoters for driving the expression of one or more cloned polynucleotide, terminators and selectable marker sequences to detest presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference. Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

### Gene Silencing

As discussed above, strategies designed to reduce, or eliminate, expression of a polynucleotide/polypeptide in a plant cell, tissue, organ, or at a particular developmental stage which/when it is normally expressed, are known as gene silencing strategies.

Gene silencing strategies may be focused on the gene itself or regulatory elements which effect expression of the transcript. “Regulatory elements” is used here in the widest possible sense and includes other genes which interact with the gene of interest.

Genetic constructs designed to decrease or silence the expression of a polynucleotide of the invention may include an antisense copy of all or part a polynucleotide described herein. In such constructs the polynucleotide is placed in an antisense orientation with respect to the promoter and terminator.

An “antisense” polynucleotide is obtained by inverting a polynucleotide or a segment of the polynucleotide so that the transcript produced will be complementary to the mRNA transcript of the gene, e.g.,

5′GATCTA 3′ (coding strand)
3′CTAGAT 5′ (antisense strand)
3′CUAGAU 5′ mRNA
5′GAUCUCG 3′ antisense RNA

Genetic constructs designed for gene silencing may also include an inverted repeat. An ‘inverted repeat’ is a sequence that is repeated where the second half of the repeat is in the complementary strand, e.g.,

5′-GATCTA . . . TAGATC-3′
3′-CTAGAT . . . ATCTAG-5′

The transcript formed may undergo complementary base pairing to form a hairpin structure. Usually a spacer of at least 3-5 bp between the repeated region is required to allow hairpin formation.

Such constructs are used in RNA interference (RNAi) approaches.

Another silencing approach involves the use of a small antisense RNA targeted to the transcript equivalent to an miRNA (Llave et al., 2002, Science 297, 2053). Use of such small antisense RNA corresponding to polynucleotide of the invention is expressly contemplated.

Transformation with an expression construct, as herein defined, may also result in gene silencing through a process known as sense suppression (e.g. Napoli et al., 1990, Plant Cell 2, 279; de Carvalho Niebel et al., 1995, Plant Cell, 7, 347). In some cases sense suppression may involve over-expression of the whole or a partial coding sequence but may also involve expression of non-coding region of the gene, such as an intron or a 5′ or 3′ untranslated region (UTR). Chimeric partial sense constructs can be used to coordinately silence multiple genes (Abbott et al., 2002, Plant Physiol. 128(3): 844-53; Jones et al., 1998, Planta 204: 499-505). The use of such sense suppression strategies to silence the target polynucleotides/genes is also contemplated.

The polynucleotide inserts in genetic constructs designed for gene silencing may correspond to coding sequence and/or non-coding sequence, such as promoter and/or intron and/or 5′ or 3′-UTR sequence, or the corresponding gene.

Preferably the insert sequence for use in a construct (e.g. an antisense, sense suppression or RNAi construct) for silencing of a target gene, comprises an insert sequence of at least 21 nucleotides in length corresponding to, or complementary, to the target gene.

Other gene silencing strategies include dominant negative approaches and the use of ribozyme constructs (McIntyre, 1996, Transgenic Res, 5, 257). Pre-transcriptional silencing may be brought about through mutation of the gene itself or its regulatory elements. Such mutations may include point mutations, frameshifts, insertions, deletions and substitutions.

Several further methods known in the art may be employed to alter, reduce or eliminate expression of a polynucleotide and/or polypeptide according to the invention. Such methods include but are not limited to Tilling (Till et al., 2003, Methods Mol Biol, 2%, 205), so called “Deletagene” technology (Li et al., 2001, Plant Journal 27(3), 235) and the use of artificial transcription factors such as synthetic zinc finger transcription factors. (e.g. Jouvenot et al., 2003, Gene Therapy 10, 513). Additionally antibodies or fragments thereof, targeted to a particular polypeptide may also be expressed in plants to modulate the activity of that polypeptide (Jobling et al., 2003, Nat. Biotechnol., 21(1), 35). Transposon tagging approaches may also be applied. Additionally peptides interacting with a polypeptide of the invention may be identified through technologies such as phase-display (Dyax Corporation). Such interacting peptides may be expressed in or applied to a plant to affect activity of a polypeptide of the invention. Use of each of the above approaches in alteration of expression of a nucleotide and/or polypeptide of the invention is specifically contemplated.

### Methods for Modifying Endogenous DNA Sequences in Plant

Methods for modifying endogenous genomic DNA sequences in plants are known to those skilled in the art. Such methods may involve the use of sequence-specific nucleases that generate targeted double-stranded DNA breaks in genes of interest. Examples of such methods for use in plants include: zinc finger nucleases (Curtin et al., 2011. Plant Physiol. 156:466-473.; Sander, et al., 2011. Nat. Methods 8:67-69.), transcription activator-like effector nucleases or “TALENs” (Cermak et al., 2011, Nucleic Acids Res. 39:e82; Mahfouz et al., 2011 Proc. Natl. Acad. Sci. USA 108:2623-2628; Li et al., 2012 Nat. Biotechnol. 30:390-392), and LAGLIDADG homing endonucleases, also termed “meganucleases” (Tzfira et al., 2012. Plant Biotechnol. J. 10:373-389).

In certain embodiments of the invention, one of these technologies (e.g. TALENs or a Zinc finger nuclease) can be used to modify one or more base pairs in a target gene to disable it, so it is no longer transcribaable and/or translatable.

Those skilled in the art will thus appreciate that there are numerous ways in which expression of target genes/polynucleotides/polypeptides can be reduced or eliminated. Any such method is included within the scope of the invention.

### Transformation Protocols

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9: 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24(1):45-51); Prunus (Ramesh et al., 2006 Plant Cell Rep. 25(8):821-8; Song and Sink 2005 Plant Cell Rep. 2006; 25(2):117-23; Gonzalez Padilla et al., 2003 Plant Cell Rep. 22(1):38-45); strawberry (Oosumi et al., 2006 Planta. 223(6):1219-30; Folta et al., 2006 Planta April 14; PMID: 16614818), rose (Li et al., 2003), Rubus (Graham et al., 1995 Methods Mol Biol. 1995; 44:129-33), tomato (Dan et al., 2006, Plant Cell Reports V25:432-441), apple (Yao et al., 1995, Plant Cell Rep. 14, 407-412) and Actinidia eriantha (Wang et al., 2006, Plant Cell Rep. 25, 5: 425-31). Transformation of other species is also contemplated by the invention. Suitable other methods and protocols are available in the scientific literature.

### Plants

The term “plant” is intended to include a whole plant, any part of a plant, propagules and progeny of a plant.

The term ‘propagule’ means any part of a plant that may be used in reproduction or propagation, either sexual or asexual, including seeds and cuttings.

The plants of the invention may be grown and either self-ed or crossed with a different plant strain and the resulting off-spring from two or more generations also form an aspect of the present invention. Preferably the off-spring retain the construct, transgene or modification according to the invention.

### General

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In certain embodiments the term “comprising” and related terms such as “comprise” and “comprises”, can be replaced with “consisting” and related terms, such as “consist” and “consists”.

### BRIEF DESCRIPTION OF THE DRAWINGS

The present invention will be better understood with reference to the accompanying drawings in which:

FIG. 1 shows that over-expression of miRNA172p reduces the size of fruit, seeds and fruit cells in transgenic ‘Royal Gala’ (RG) plant TRG3. The photographs show a mature fruit (a), mature seeds (b), and thin (10 μm) sections of mature fruit cortex tissues (c) of RG, TRG3 and crabapple M. sieboldii ‘Aotea’ from left to right. The graphs on the far right panel show mean fruit weight (n=20), mean weight of 10 seeds (n=10) and mean fruit cortex cell area (n=20) for the fruit from the three plants. The error bars in the graphs represent standard deviation.

FIG. 2 shows the determination of the relationship between the cafs allele of miRNA172p and Malus fruit size. a, Fruit of M.×domestica (Dom), M. sieversii (Sie), M. onentalis (On), M. sylvestris (Syl) and M. baccata (Bac). b, The sequences specific to the 2 kb promoter region and 2 kb pri-miRNA for 12 accessions of M. baccata defined as CAFS allele shown in black, and 64 accessions of M. domestica, M. sieversii, M. orientalis and M. sylvestris defined as cafs allele are shown in red, ins: insertion, del: deletion, TE ins: transposable element insertion, NS: not sequenced. Position of the mature miRNA172p is indicated. c, Box plot of fruit size distribution of 91 cafs/cafs and 68 CAFS/cafs progeny plants of RG×A689-24. Whiskers extend from the lower and upper quartile to the minimum and maximum respectively. From lower quartile to median and from median to upper quartile are filled in by different colours. d, The pri-miRNA172p expression levels were reduced in four cafs/cafs plants compared to two CAFS/cafs plants (relative level, error bars represent the standard deviation of three PCR reactions).

FIG. 3 shows altered phenotypes of transgenic ‘Royal Gala’ over-expressing miRNA172p. a, b, c, d, Flowers of wild-type ‘Royal Gala’ (a), transgenic ‘Royal Gala’ TRG3 (b), and TRG5 (c, d). Some petals were removed to show partial sepal to petal transformation (b) and leaves removed to show ovaries (d). e, f, g, Shown are the same aged (two-year old) trees of wild-type ‘Royal Gala’ (e), TRG5 (f) and TRG6 (g) grown under the same conditions.

FIG. 4 shows over-expression of miRNA172p reduces hypanthium and fruit cortex width and fruit cell size. a, b, c, The photographs show thin (10 μm) sections of hypanthium at full-bloom stage (a), fruit cortex at 2-weeks (b) and 5-weeks (c) following pollination of wild-type ‘Royal Gala” (RG), transgenic ‘Royal Gala” TRG3 and a crabapple M. sieboldii ‘Aotea’. Graphs on the right hand side panels show mean hypanthium and cortex tissue width and mean cell area (n=20). The error bars in the graphs represent standard deviation.

FIG. 5 shows a phylogenetic analysis of the 4 kb genomic region of miRNA172p. Rooted Neighbour-joining phylogenetic tree constructed using genomic sequence of miRNA172p from 12 accessions of Malus baccata (Bac) and 64 accessions of M.×x domestica (Dom), M. sieversii (Sie), M. orientalis (Ori) and M. sylvestris (Syl). The number for each sequence corresponds to the sequence number given in Supplementary Table 1. Sequences from two pear species, Pyrus communis (Pc) and P. bretschneideri (Pb) were used as an outgroup

FIG. 6 shows the 3′ region of pri-miRNA172p sequence contains a transposable element (TE). The TE is shown in red, its 18 bp imperfect inverted terminal repeats are indicated by arrows, and its target site duplicated direct repeats are underlined in blue. The positions of miRNA172 and PCR primers used in this study are also indicated. The sequence is from GenBank Accession No EG999280 and is shown in SEQ ID NO:47.

FIG. 7 shows the TE in pri-miRNA172p belongs to a MITE-type transposon family. The TE sequences and their target site duplicated sequences from six apple genes are aligned. The duplicated target site sequences are underlined and imperfect inverted terminal repeats are indicated by arrows. GenBank Accession Nos: MdmiRNA172p=EG999280 (SEQ ID NO:48); MdOmt2=DQ886019 (SEQ ID NO:49); MdACS1=U89156 (SEQ ID NO:50); MdAGL-1=GU56825 (SEQ ID NO:51); MsS46-RNase=EU419860 (SEQ ID NO:52); MdRfa2=AB073704 (SEQ ID NO:53).

FIG. 8 shows Fruit weight quantitative trait locus (QTL) analysis in the ‘Royal Gala’×A689-24 segregating population. a, The position of the CAFS allele on linkage group (LG) 11 of A689-24 is presented alongside the intervals for fruit weight QTLs in three consecutive years (2006 to 2008). B, LOD score, position and percentage of phenotypic variation explained by the QTL.

FIG. 9 shows description of 153 accessions from 36 Malus species sequenced and allelotyped at CAFS locus tested in this study

FIG. 10 shows the alignment of mature miRNA172 sequences from seven plant species. ath, Arabidopsis thaliana; mdm, Malus×domestica, ppe, Prunus persica; csi, Citrus sinensis; sly, Solanum lycopersicum; vvi, Vitis vinifera; cpa, Carica papaya. The sequences are: ath-miR172b=SEQ ID NO:54; ath-miR172c=SEQ ID NO:55; ath-miR172d=SEQ ID NO:56; ath-miR172a=SEQ ID NO:57; ath-miR172e=SEQ ID NO:58; mdm-miR172d=SEQ ID NO:59; mdm-miR172e=SEQ ID NO:60; mdm-miR172j=SEQ ID NO:61; mdm-miR172g=SEQ ID NO:62; mdm-miR172a=SEQ ID NO:63; mdm-miR172k=SEQ ID NO:64; mdm-miR172f=SEQ ID NO:65; mdm-miR172o=SEQ ID NO:66; mdm-miR172l=SEQ ID NO:67; mdm-miR172n=SEQ ID NO:68; mdm-miR172b=SEQ ID NO:69; mdm-miR172c=SEQ ID NO:71; mdm-miR172i=SEQ ID NO:72; mdm-miR172h=SEQ ID NO:73; ppe-miR172d=SEQ ID NO:74; ppe-miR172a-3p=SEQ ID NO:75; ppe-miR172b=SEQ ID NO:76; ppe-miR172c=SEQ ID NO:77; csi-miR172a-3p=SEQ ID NO:78; csi-miR172c=SEQ ID NO:79; csi-miR172b=SEQ ID NO:80; sly-miR172b=SEQ ID NO:81; sly-miR172a=SEQ ID NO:82; vvi-miR172a=SEQ ID NO:83; vvi-miR172c=SEQ ID NO:84; vvi-miR172b=SEQ ID NO:85; cpa-miR172a=SEQ ID NO:86; cpa-miR172b=SEQ ID NO:87; Consensus (of sequences of SEQ ID NO: 54 to 87)=SEQ ID NO:88.

### EXAMPLES

The invention will now be illustrated with reference to the following non-limiting examples.

It is not the intention to limit the scope of the invention to the present example only. As would be appreciated by a skilled person in the art, many variations are possible without departing from the scope of the invention.

### Example 1: Altering Apple Fruit Size

### Summary

Developing an understanding of the molecular basis for the genetic control of domestication traits can guide modern breeding programs. In annual crops, more than 20 genes underlying domestication traits have been characterised, revealing that specific genetic mutations affecting these traits have been selected during domestication until they are fixed. However, there is little genetic information on domestication in perennial tree crops⁽¹⁾.

Here the applicants show that a transposon insertional mutation in a miRNA172 gene (which reduces expression of miRNA172) is strongly associated with large apple fruit size in segregating progenies, and over-expression of miRNA172 resulted in more than 60-fold reduction in fruit weight in transgenic ‘Royal Gala’, coupled with a reduction in cell division and expansion in fruit tissues.

### Introduction

Fruit crop domestication is typically associated with a dramatic increase in fruit size. Despite its fundamental and applied importance, the molecular genetics underlying this important agronomic trait is still poorly understood, particularly in perennial species.

The cultivated apple (Malus×domestica) has both cultural and economic significance, being the second fruit tree crop in terms of worldwide production. Although most wild apple species bear bitter, small fruits (<1 cm in diameter) termed crabapples, some species produce relatively large fruit (>1 cm), and it is these species (M. sieversii, M. sylvestris and M. orientalis) that have contributed to the genome of the cultivated apple. Malus sieversii in particular, the primary progenitor of the cultivated apple, has fruit up to 8 cm in diameter, which is still not as large as cultivated apples.

### Results

The applicants identified microRNA172 (miRNA172) as a possible candidate for the regulation of apple fruit size. miRNA172 inhibits the translation of a subfamily of Apetalla2 (AP2) genes⁽¹⁶⁾ that govern floral organ development⁽¹⁷⁾ and floral organ size⁽¹⁸⁾ in Arabidopsis. Fifteen miRNA172 genes (a-o) have been predicted from the genome sequences⁽²⁾ and one gene (miRNA172p) from EST sequences⁽³⁾ of the cultivated apple, but only the expression of miRNA172p has been confirmed to date⁽¹⁹⁾.

The applicants surprisingly found that miRNA172p over-expression resulted in the reversion of cultivated apple fruit to crabapple size, in addition to causing other altered phenotypes in transgenic ‘Royal Gala’ (RG) apple plants (Table 2).

TABLE 2
Descriptions of ‘Royal Gala’ apple transgenic plants
developed using a CaMV35S-pri-miRNA172p gene construct

| | Relative | | | | | |
|---|---|---|---|---|---|---|
| | Presence of expression level | | | | | Fruit |
| Plant | Presence | miRNA172 of | miRNA172 | Plant | | weight (g) |
| ID | of NPTII^{a} | transgene^{b} | (mean ± SD)^{c} | height | Flower | (mean ± SD)^{d} |
| RG | No | No | 1.00 ± 0.47 | normal | normal | 127.9 ± 39.9 |
| TRG1 | Yes | No | 0.80 ± 0.0.37 | normal | normal | 127.9 ± 21.1 |
| TRG2 | Yes | No | 1.19 ± 0.68 | normal | normal | 110.9 ± 630.6 |
| TRG3 | Yes | Yes | 15.80 ± 3.55 | normal | partial sepal | 2.0 ± 0.4 |
| | | | | | to petal | |
| | | | | | conversion | |
| TRG4 | Yes | Yes | 20.27 ± 7.37 | normal | carpel only | No fruit |
| TRG5 | Yes | Yes | 22.99 ± 8.67 | semi-dwarf | carpel only | No fruit |
| TRG6 | Yes | Yes | 23.72 ± 0.39 | dwarf | no flower | No fruit |

^{a}PCR analysis using primers binding to NPTII gene
^{b}PCR analysis using primers binding the CaMV35S promoter and pri-miRNA172p
^{c}Stem-loop RT-PCR miRNA assay, mean and standard deviation (SD) of two leaf and two flower biological samples. For TRG6, only two leaf samples were used, as no flowers were produced.
^{d}Mean and SD of 20 fruit.

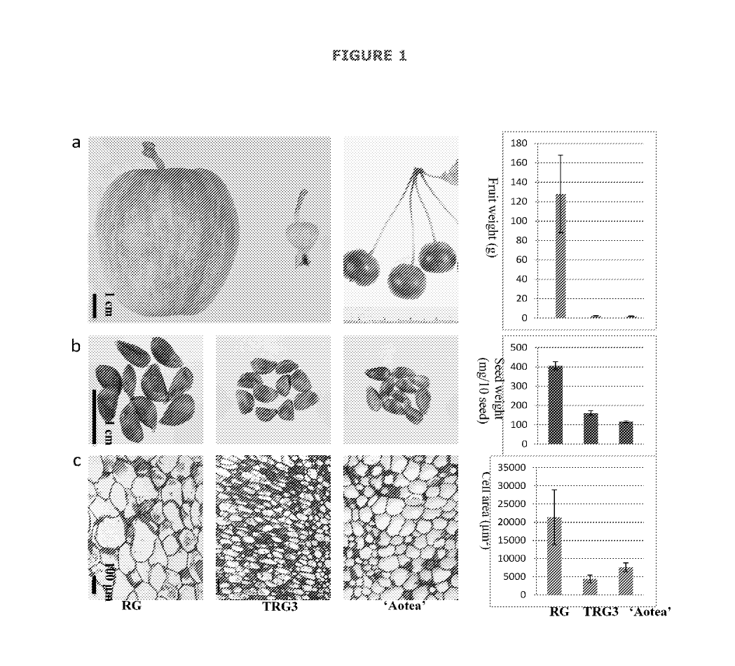

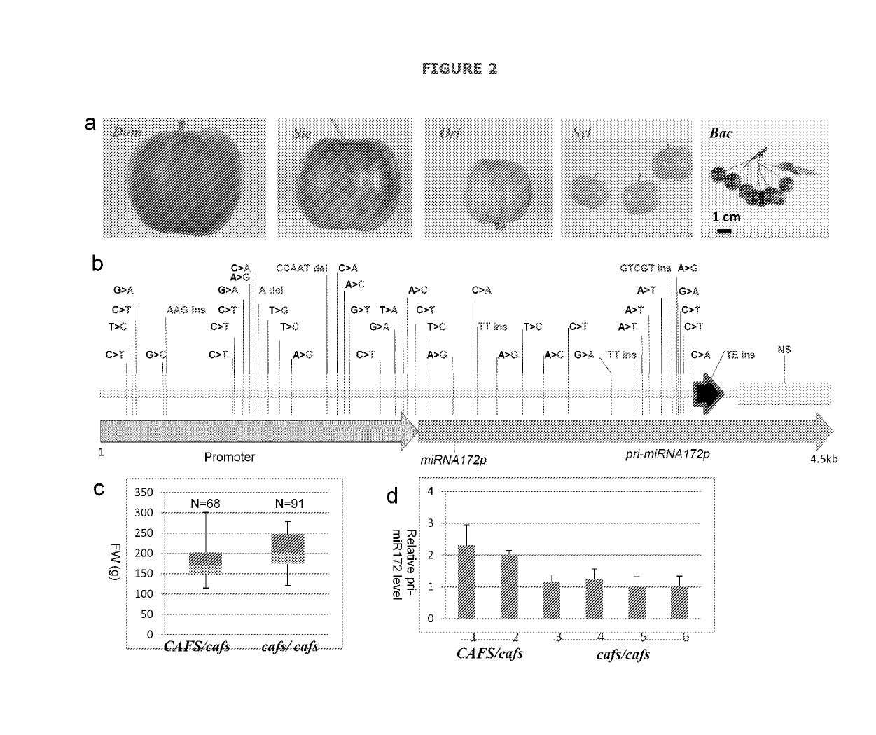

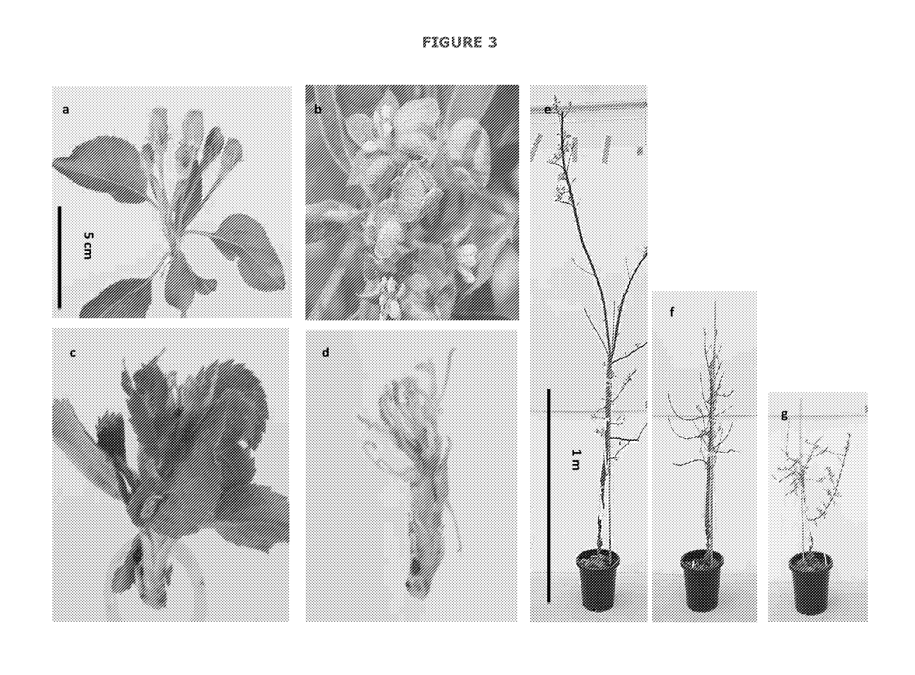

The transgenic plant TRG3 that over-expressed miRNA172p 15-fold, exhibited significantly smaller fruit and seeds than the RG control (FIG. 1a, b) and had some flowers with sectors of sepals converted to petal identity (FIG. 3). Plants TRG4 and TRG5, with 20- and 23-fold over-expression of miRNA172p respectively, exhibited greater changes in phenotype, including flowers consisting entirely of carpel tissues, with no sepals, petals, or stamens (FIG. 3c, d) and failed to produce any fruit after hand-pollination. These phenotypes for altered floral organ development were similar to those reported following miRNA172 over-expression in other species^{(4 and 5)}. TRG5 was a semi-dwarfed plant (FIG. 2f). With 24-fold over-expression of miRNA172 TRG6 exhibited the severest alteration of phenotype, not only being dwarfed (FIG. 2g), but also producing no flowers or fruit (Table 2).

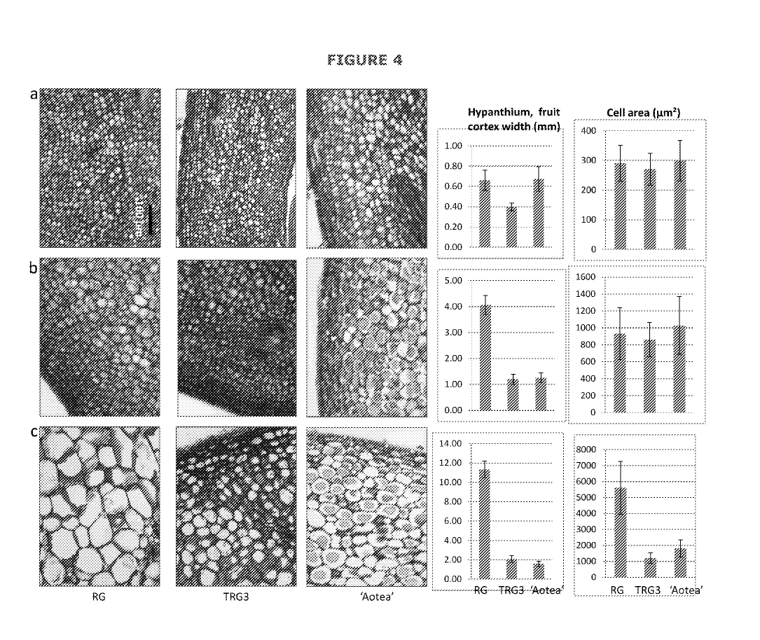

The key developmental difference between the large fruit of domesticated apple and smaller crabapples has been reported to be the reduction of fruit cell number and cell size in the latter⁽⁶⁾. TRG3 had fewer cells than RG in the hypanthium and in two-week old fruit, as it displayed significantly thinner hypanthium at full bloom and thinner fruit cortex tissue than RG at two weeks, but exhibited similar cell sizes (FIG. 4). TRG3 fruit cortex tissues displayed reduced cell size compared with RG from five weeks to maturity. This developmental data indicate that the elevated miRNA172p expression inhibited cell division and cell expansion at the early and late stages of fruit development, respectively. The crabapple M. sieboldii ‘Aotea’ exhibited a similar reduction of fruit cell number and size as did TRG3 (FIG. 1c and FIG. 4b, c). Given the similarity in fruit size, fruit cell number and size between TRG3 and crabapples, the applicants postulated that a mutated allele of miRNA172p with reduced expression may be responsible for the increase in fruit size in domesticated apple.

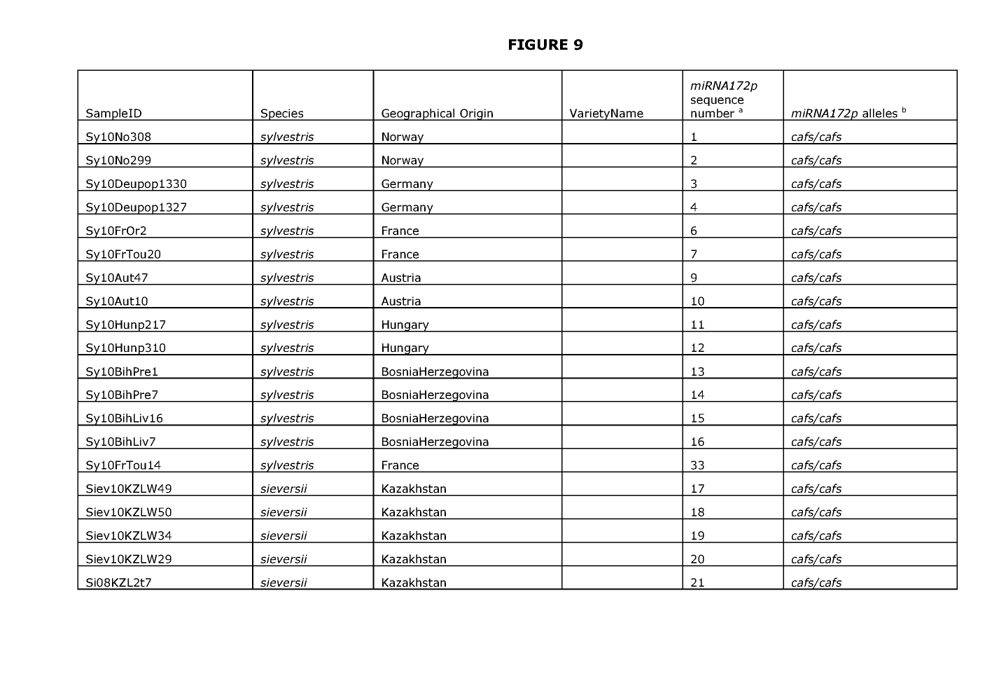

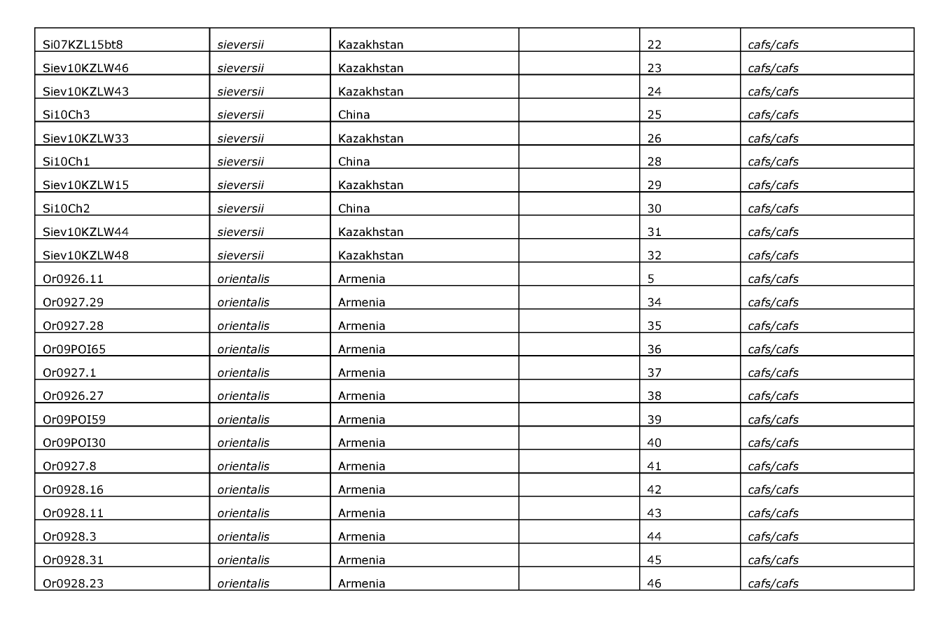

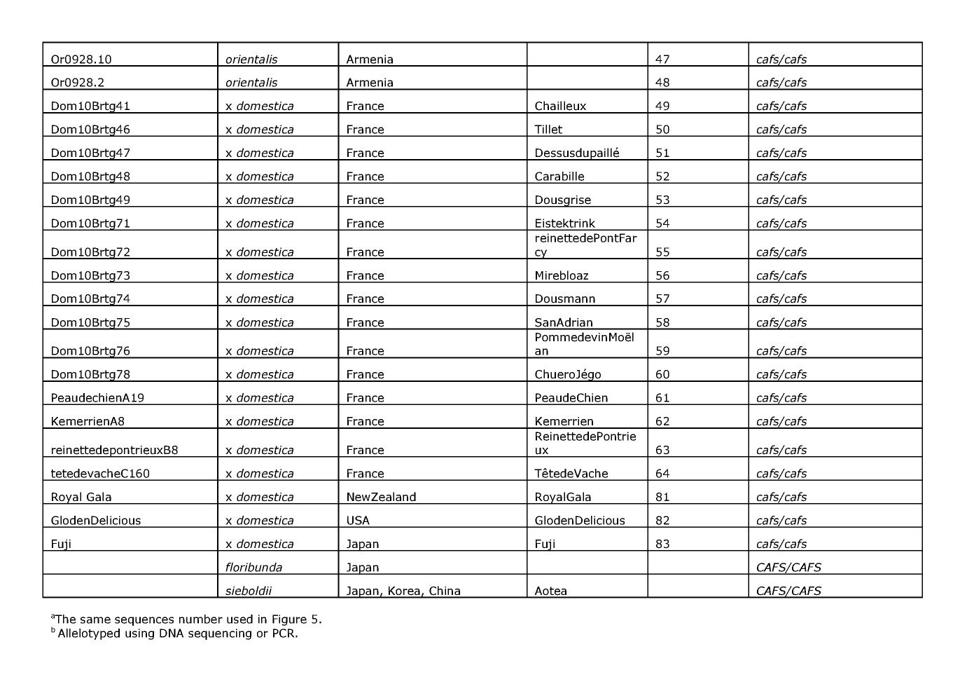

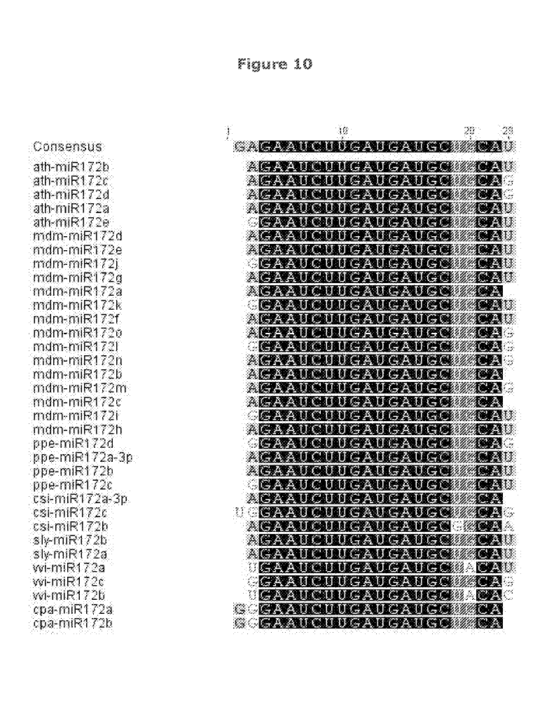

To test this hypothesis, the applicants sequenced DNA amplicons (up to 3957 bp) of miRNA172p from 64 accessions of four apple species that produce relatively large fruit (M.×domestica, M. sieversii, M. orientalis and M. sylvestris) and 12 accessions of a crabapple species (M. baccata) that bears very small fruits (FIG. 2a, FIG. 9 Table 3).

TABLE 3
Distribution of CAFS and cafs alleles in the genus Malus

| | | | | | | | Fruit | |
|---|---|---|---|---|---|---|---|---|
| | | | N. | CAFS/ | CAFS/ | cafs/ | diameter | |
| Section^{a} | Series^{a} | Species^{a} | tested | CAFS | cafs | cafs | (cm) | Ref^{b} |
| Malus | Malus | ×domestica | 19 | | | 19 | 6.0-10 | 24 |
| | | sieversii | 15 | | | 15 | 3.0-8.0 | FOC |
| | | orientalis | 15 | | | 15 | 2.0-4.0 | 24 |
| | | sylvestris | 15 | | | 15 | 1.0-3.0 | 24 |
| | Baccatae | baccata | 12 | 12 | | | 0.8-1 | FOC |
| Sorbomalus | Sieboldianae | floribunda | 1 | 1 | | | NA | NA |
| | | sieboldii | 1 | 1 | | | 0.6-0.8 | FOC |
| Total | | | 78 | 14 | | 64 | | |

^{a}as classified by Phipps et al⁽⁷⁾
^{b}References for fruit diameter.
FOC: Flora of China http://foc.eflora.cn/, NA: no data available;
HR: Horticultural Reviews, Wild Apple and Fruit Trees of Central Asia, RHS: Royal Horticultural Society, http://apps.rhs.orq.uk/plantselector/plant?plantid=1259, USDA: https://plants.usda.gov/java/

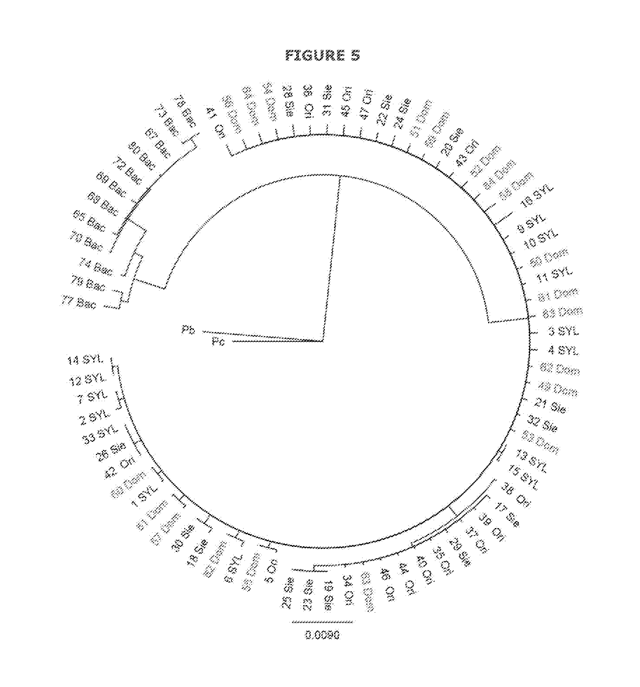

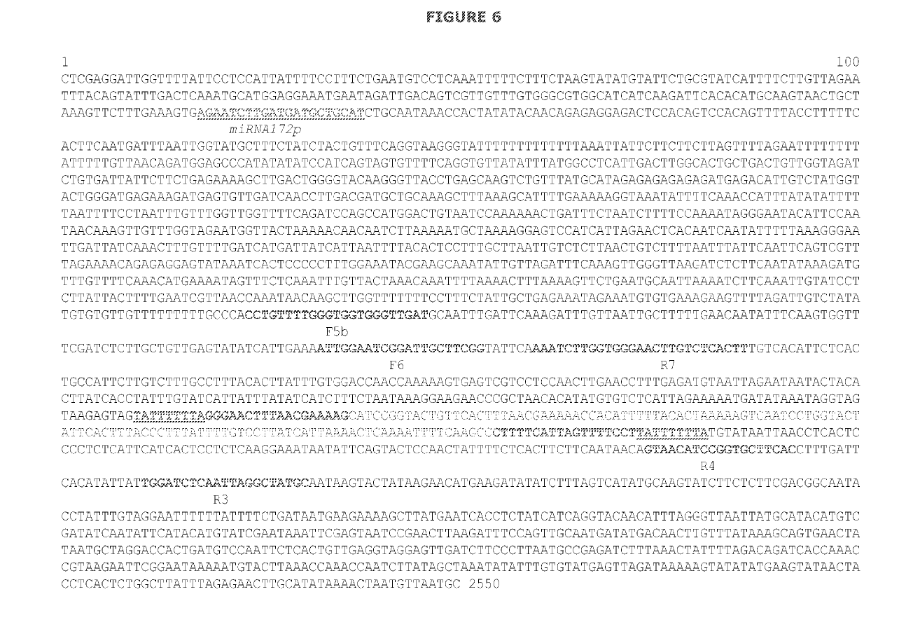

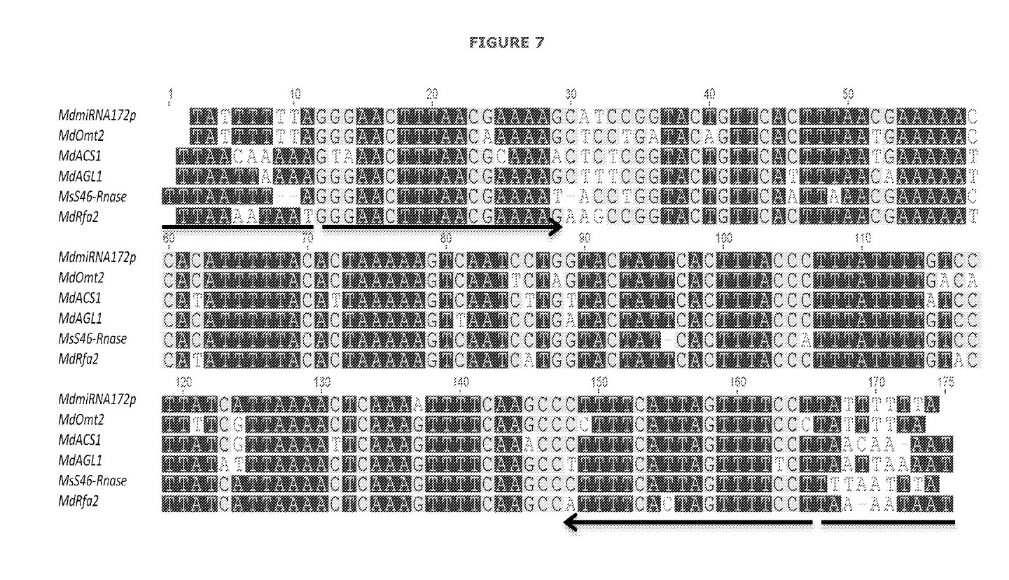

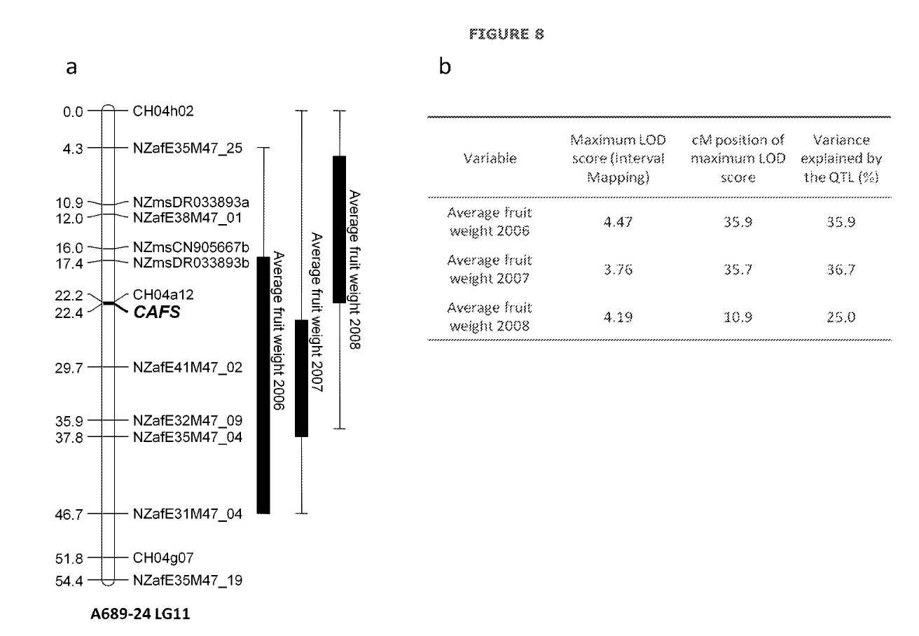

A phylogenetic tree derived from these sequences showed that all 12 M. baccata accessions clustered together, and that the accessions of the other four species formed a separate clade, with no further phylogenetic structure according to species (FIG. 5). The two-clade structure was due to six small indels (1 to 5 bp) and 38 SNPs (FIG. 2b) between M. baccata and the four large fruited species. In addition, the four large fruited species exhibited a transposable element (TE) insertion in the 3′ end of pri-miRNA172p (FIG. 2b and FIG. 6), that was absent in the sequences from M. baccata. The 154 bp long TE belonged to a MITE-type transposon family (FIG. 7). As the TE can form stem-loop structures and alter gene expression²⁵, the applicants hypothesized that the presence of the TE may reduce the expression level of miRNA172p. The applicants named the miRNA172p locus as CrabApple Fruit Size and its wild type and transposon insertion alleles as CAFS and cafs respectively.

To confirm the role of the cafs allele in apple fruit size evolution, the applicants further allelotyped the miRNA172p locus of two crableapple species, M. floribunda and M. sieboldii, using PCR analysis (FIG. 9). These two species are CAFS homozygous (Table 3). Together with the DNA sequencing data showed above, it is clear that the cafs allele is associated with large fruit and CAFS allele associated with small fruit.

The association between the cafs allele and a large fruit size was confirmed by analysing a segregating progeny from a RG (cafs/cafs)×A689-24 (CAFS/cafs) cross (Table 4).

TABLE 4
Description of 159 progeny plants of
RG × A689-24 tested in this study

| | 2008 | 2007 | 2006 | 3-year | |
|---|---|---|---|---|---|
| Leaf | average | average | average | average | |
| sample | FW | FW | FW | FW | miRNA172p |
| ID | (g) | (g) | (g) | (g) | alleles |
| M871 | 145.33 | 138.00 | 120.00 | 134.44 | cafs/cafs |
| AM864 | 124.44 | 120.00 | 160.00 | 134.81 | cafs/cafs |
| An231 | 171.54 | 120.00 | 135.00 | 142.18 | cafs/cafs |
| AJ347 | 127.78 | 150.00 | 195.00 | 157.59 | cafs/cafs |
| An216 | 145.33 | 140.00 | 190.00 | 158.44 | cafs/cafs |
| An195 | 143.33 | 150.00 | 190.00 | 161.11 | cafs/cafs |
| AM860 | 121.11 | 140.00 | 225.00 | 162.04 | cafs/cafs |
| AJ411 | 173.33 | 150.00 | 180.00 | 167.78 | cafs/cafs |
| AN321 | 162.86 | 180.00 | 170.00 | 170.95 | cafs/cafs |
| AN300 | 183.33 | 165.00 | 165.00 | 171.11 | cafs/cafs |
| An242 | 176.25 | 180.00 | 160.00 | 172.08 | cafs/cafs |
| AJ429 | 148.33 | 195.00 | 180.00 | 174.44 | cafs/cafs |
| AM884 | 169.66 | 165.00 | 195.00 | 176.55 | cafs/cafs |
| AM875 | 177.73 | 210.00 | 150.00 | 179.24 | cafs/cafs |
| AJ423 | 194 | 150.00 | 210.00 | 184.67 | cafs/cafs |
| AM843 | 166.84 | 195.00 | 200.00 | 187.28 | cafs/cafs |
| AN322 | 204.76 | 165.00 | 195.00 | 188.25 | cafs/cafs |
| AN313 | 229.44 | 150.00 | 195.00 | 191.48 | cafs/cafs |
| An215 | 186.67 | 200.00 | 190.00 | 192.22 | cafs/cafs |
| AN274 | 205.83 | 195.00 | 180.00 | 193.61 | cafs/cafs |
| AN279 | 176.5 | 255.00 | 150.00 | 193.83 | cafs/cafs |
| An230 | 196 | 210.00 | 180.00 | 195.33 | cafs/cafs |
| AJ349 | 200 | 195.00 | 195.00 | 196.67 | cafs/cafs |
| AN298 | 196.11 | 187.50 | 210.00 | 197.87 | cafs/cafs |
| AM885 | 210 | 195.00 | 195.00 | 200.00 | cafs/cafs |
| AJ428 | 225 | 220.00 | 160.00 | 201.67 | cafs/cafs |
| An207 | 195.71 | 220.00 | 190.00 | 201.90 | cafs/cafs |
| AN308 | 178 | 230.00 | 200.00 | 202.67 | cafs/cafs |
| AJ409 | 199.52 | 210.00 | 200.00 | 203.17 | cafs/cafs |
| AN283 | 232.27 | 230.00 | 150.00 | 204.09 | cafs/cafs |
| AN280 | 201.56 | 230.00 | 190.00 | 207.19 | cafs/cafs |
| AN316 | 198 | 220.00 | 210.00 | 209.33 | cafs/cafs |
| AJ341 | 205.83 | 180.00 | 250.00 | 211.94 | cafs/cafs |
| AM796 | 180 | 195.00 | 270.00 | 215.00 | cafs/cafs |
| AN320 | 226 | 240.00 | 195.00 | 220.33 | cafs/cafs |
| AM850 | 218.33 | 240.00 | 220.00 | 226.11 | cafs/cafs |
| An222 | 223.33 | 225.00 | 240.00 | 229.44 | cafs/cafs |
| AN335 | 248.52 | 270.00 | 180.00 | 232.84 | cafs/cafs |
| AJ408 | 253.13 | 220.00 | 240.00 | 237.71 | cafs/cafs |
| AN318 | 192.73 | 255.00 | 270.00 | 239.24 | cafs/cafs |
| AM887 | 258 | 230.00 | 255.00 | 247.67 | cafs/cafs |
| AM886 | 224.62 | 250.00 | 270.00 | 248.21 | cafs/cafs |
| AN304 | 267.33 | 270.00 | 225.00 | 254.11 | cafs/cafs |
| AM846 | 236.5 | 260.00 | 270.00 | 255.50 | cafs/cafs |
| AM895 | 234.29 | 270.00 | 270.00 | 258.10 | cafs/cafs |
| AM881 | 267.69 | 270.00 | 240.00 | 259.23 | cafs/cafs |
| AM899 | 238.33 | 270.00 | 270.00 | 259.44 | cafs/cafs |
| AM865 | 283.48 | 240.00 | 255.00 | 259.49 | cafs/cafs |
| AN297 | 247.5 | 270.00 | 270.00 | 262.50 | cafs/cafs |
| AJ343 | 278.67 | 240.00 | 270.00 | 262.89 | cafs/cafs |
| AM892 | 320 | 200.00 | 270.00 | 263.33 | cafs/cafs |
| AN281 | 280.71 | 250.00 | 270.00 | 266.90 | cafs/cafs |
| AM896 | 287.14 | 260.00 | 255.00 | 267.38 | cafs/cafs |
| AJ431 | 292.22 | 240.00 | 270.00 | 267.41 | cafs/cafs |
| AN306 | 262.92 | 270.00 | 270.00 | 267.64 | cafs/cafs |
| AN301 | 322.22 | 225.00 | 270.00 | 272.41 | cafs/cafs |
| An233 | 296.32 | 260.00 | 270.00 | 275.44 | cafs/cafs |
| AM891 | 294.55 | 270.00 | 270.00 | 278.18 | cafs/cafs |
| AJ327 | 241.5 | 220 | 225 | 228.8 | cafs/cafs |
| AJ330 | 94 | 195 | 225 | 171.3 | cafs/cafs |
| AJ332 | . | 130 | 210 | 170.0 | cafs/cafs |
| AJ351 | 128.82 | 120 | 130 | 126.3 | cafs/cafs |
| AJ353 | 213.33 | 135 | 165 | 171.1 | cafs/cafs |
| AJ354 | 224.5 | 180 | 210 | 204.8 | cafs/cafs |
| AJ371 | 195 | . | 180 | 187.5 | cafs/cafs |
| AJ372 | 161.74 | 140 | 150 | 150.6 | cafs/cafs |
| AJ374 | 190.83 | 180 | 180 | 183.6 | cafs/cafs |
| AJ380 | 194.29 | 200 | 210 | 201.4 | cafs/cafs |
| AJ381 | 185 | 195 | 240 | 206.7 | cafs/cafs |
| AJ383 | 287.5 | . | 270 | 278.8 | cafs/cafs |
| AJ391 | 236.8 | 270 | 270 | 258.9 | cafs/cafs |
| AJ398 | 172 | . | . | 172.0 | cafs/cafs |
| AJ405 | 204.44 | 210 | 180 | 198.1 | cafs/cafs |
| AJ406 | 285.24 | 240 | 150 | 225.1 | cafs/cafs |
| AJ421 | 181.88 | 180 | 190 | 184.0 | cafs/cafs |
| AM772 | . | 150 | 210 | 180.0 | cafs/cafs |
| AM776 | 150 | 240 | . | 195.0 | cafs/cafs |
| AM787 | 273.43 | 260 | 270 | 267.8 | cafs/cafs |
| AN264 | 211.76 | 200 | 255 | 222.3 | cafs/cafs |
| AN265 | 224.17 | 120 | 120 | 154.7 | cafs/cafs |
| AN269 | 126 | . | 150 | 138.0 | cafs/cafs |
| AN270 | 219 | 210 | . | 214.5 | cafs/cafs |
| AN271 | 187.5 | 190 | 225 | 200.8 | cafs/cafs |
| AN272 | 236.43 | 255 | 270 | 253.8 | cafs/cafs |
| AN288 | 165.71 | 180 | 165 | 170.2 | cafs/cafs |
| AN290 | 113.04 | 130 | 120 | 121.0 | cafs/cafs |
| AN293 | 126.67 | . | . | 126.7 | cafs/cafs |
| AN323 | 285 | 270 | 270 | 275.0 | cafs/cafs |
| AN328 | 185.19 | 150 | 157.5 | 164.2 | cafs/cafs |
| AN331 | 192.5 | 230 | 255 | 225.8 | cafs/cafs |
| Y123 | 239.3 | 250 | 270 | 253.1 | cafs/cafs |
| AN278 | 113.53 | 110.00 | 120.00 | 114.51 | CAFS/cafs |
| AN310 | 115.88 | 130.00 | 135.00 | 126.96 | CAFS/cafs |
| AM797 | 131.82 | 120.00 | 135.00 | 128.94 | CAFS/cafs |
| AM756 | 138.33 | 105.00 | 150.00 | 131.11 | CAFS/cafs |
| AJ432 | 123.5 | 135.00 | 140.00 | 132.83 | CAFS/cafs |
| AM853 | 174.71 | 127.50 | 120.00 | 140.74 | CAFS/cafs |
| An237 | 143.18 | 130.00 | 150.00 | 141.06 | CAFS/cafs |
| AM851 | 136.92 | 150.00 | 140.00 | 142.31 | CAFS/cafs |
| AM854 | 132.31 | 150.00 | 150.00 | 144.10 | CAFS/cafs |
| An210 | 150.8 | 126.00 | 165.00 | 147.27 | CAFS/cafs |
| AM878 | 123.08 | 150.00 | 180.00 | 151.03 | CAFS/cafs |
| AM834 | 154.38 | 150.00 | 150.00 | 151.46 | CAFS/cafs |
| AN303 | 172.86 | 165.00 | 120.00 | 152.62 | CAFS/cafs |
| AM882 | 195.85 | 150.00 | 135.00 | 160.28 | CAFS/cafs |
| AN302 | 172.86 | 180.00 | 135.00 | 162.62 | CAFS/cafs |
| AN334? | 145.33 | 135.00 | 210.00 | 163.44 | CAFS/cafs |
| AN312 | 174 | 180.00 | 140.00 | 164.67 | CAFS/cafs |
| AN315 | 167.41 | 160.00 | 170.00 | 165.80 | CAFS/cafs |
| AM845 | 202.22 | 150.00 | 150.00 | 167.41 | CAFS/cafs |
| An239 | 167.31 | 160.00 | 180.00 | 169.10 | CAFS/cafs |
| AJ427 | 178.55 | 150.00 | 180.00 | 169.52 | CAFS/cafs |
| AJ430 | 197.39 | 195.00 | 120.00 | 170.80 | CAFS/cafs |
| AJ339 | 170 | 210.00 | 135.00 | 171.67 | CAFS/cafs |
| AN333 | 126.36 | 210.00 | 180.00 | 172.12 | CAFS/cafs |
| An234 | 138 | 160.00 | 225.00 | 174.33 | CAFS/cafs |
| An220 | 183.18 | 140.00 | 210.00 | 177.73 | CAFS/cafs |
| AJ348 | 143.33 | 150.00 | 240.00 | 177.78 | CAFS/cafs |
| AM744 | 157.78 | 170.00 | 210.00 | 179.26 | CAFS/cafs |
| AJ425 | 165 | 195.00 | 180.00 | 180.00 | CAFS/cafs |
| AM757 | 228 | 120.00 | 210.00 | 186.00 | CAFS/cafs |
| AM793 | 209.44 | 210.00 | 150.00 | 189.81 | CAFS/cafs |
| AM838 | 180.63 | 180.00 | 210.00 | 190.21 | CAFS/cafs |
| AM795 | 196.67 | 210.00 | 195.00 | 200.56 | CAFS/cafs |
| AN277 | 172.5 | 210.00 | 240.00 | 207.50 | CAFS/cafs |
| AM898 | 229.03 | 170.00 | 225.00 | 208.01 | CAFS/cafs |
| AM862 | 217.22 | 210.00 | 210.00 | 212.41 | CAFS/cafs |
| An223 | 215.77 | 195.00 | 230.00 | 213.59 | CAFS/cafs |
| AN275 | 225.71 | 240.00 | 180.00 | 215.24 | CAFS/cafs |
| AM841 | 211 | 195.00 | 240.00 | 215.33 | CAFS/cafs |
| AM868 | 224.29 | 220.00 | 210.00 | 218.10 | CAFS/cafs |
| AN307 | 241.43 | 240.00 | 180.00 | 220.48 | CAFS/cafs |
| AM894 | 232.5 | 230.00 | 200.00 | 220.83 | CAFS/cafs |
| AN311 | 244.21 | 210.00 | 210.00 | 221.40 | CAFS/cafs |
| An221 | 245.71 | 250.00 | 270.00 | 255.24 | CAFS/cafs |
| AJ321 | 170 | 160 | 160 | 163.3 | CAFS/cafs |
| AJ328 | 121.9 | 110 | 135 | 122.3 | CAFS/cafs |
| AJ329 | 199.41 | 150 | 240 | 196.5 | CAFS/cafs |
| AJ352 | 362.86 | 270 | 270 | 301.0 | CAFS/cafs |
| AJ357 | 126.15 | 120 | 135 | 127.1 | CAFS/cafs |
| AJ360 | 216.25 | 255 | 270 | 247.1 | CAFS/cafs |
| AJ362 | 230 | 135 | 180 | 181.7 | CAFS/cafs |
| AJ368 | 123.33 | 180 | . | 151.7 | CAFS/cafs |
| AJ379 | 259.6 | 180 | 270 | 236.5 | CAFS/cafs |
| AJ387 | 273 | 230 | 255 | 252.7 | CAFS/cafs |
| AM758 | 227.86 | 195 | 240 | 221.0 | CAFS/cafs |
| AM770 | 123.04 | 130 | 157.5 | 136.8 | CAFS/cafs |
| AN266 | 176.43 | 170 | 170 | 172.1 | CAFS/cafs |
| AN268 | 159 | 160 | 150 | 156.3 | CAFS/cafs |
| AN284 | 274.55 | 220 | 210 | 234.9 | CAFS/cafs |
| AN285 | 142.5 | 150 | . | 146.3 | CAFS/cafs |
| AN287 | 134.14 | 130 | 165 | 143.0 | CAFS/cafs |
| AN291 | 136.25 | 130 | 135 | 133.8 | CAFS/cafs |
| AN324 | 132.5 | 120 | 120 | 124.2 | CAFS/cafs |
| AN327 | 114.78 | 135 | 200 | 149.9 | CAFS/cafs |
| AN332 | 145 | 150 | 135 | 143.3 | CAFS/cafs |
| Y127 | 194.85 | 200 | 180 | 191.6 | CAFS/cafs |
| Y128 | 145.36 | 140 | 180 | 155.1 | CAFS/cafs |
| Y138 | 192.76 | 190 | 180 | 187.6 | CAFS/cafs |

Ninety one cafs/cafs and 68 CAFS/cafs plants displayed significantly different (P=4.3×10⁻⁶) three-year average fruit weights of 206.97 g and 176.20 g, respectively, with the CAFS locus explaining 21% of the fruit weight variation (Table 5 and FIG. 2c).

TABLE 5
Association analysis of cafs allele and fruit weight
in progeny of RG (cafs/cafs) × A689-24 (CAFS/cafs)

| | | Three-year | | |
|---|---|---|---|---|
| | | average FW | | Intra-class |
| Genotype | Count | (g) | P-value^{a} | correlation ^{b} |
| cafs/cafs | 91^{c} | 206.97 | 4.3 × 10⁻⁶ | 21% |
| CAFS/cafs | 68^{c} | 176.2 | | |

^{a}Single factor ANOVA analysis
^{b} Calculated as V_{Between}_{_{—}}_{gentyotes}/(V_{Between}_{_{—}}_{gentyotes} + V_{Within})
^{c}The observed genotype counts fit a 1:1 segregation ratio as demonstrated using the Chi-squared test.

In this segregating population, the CAFS allele mapped within the 95% confidence interval of a fruit size QTL on Linkage Group 11 of A689-24, over three consecutive years (FIG. 8). Quantitative PCR analyses of cDNA from RNA of two CAFS/cafs and four cafs/cafs plants showed that the pri-miRNA172p level was reduced approximately two-fold in cafs/cafs plants (FIG. 2d). The applicant's data shows that CAFS underlies a major QTL for apple fruit size and the presence of the homozygous cafs allele results in large fruit, due to a reduction in miRNA172p transcript accumulation. CAFS however does not account for all fruit size variation and must act in association with other fruit size QTLs in M.×domestica⁽⁸⁾.

The applicant's results indicate that the cafs allele was under selection prior to domestication. The nucleotide diversities (n value) of the cafs allele in M.×domestica and the three closest wild species (M. sieversii, M. orientalis and M. sylvestris), were significantly lower than those of the CAFS allele in M. baccata and of 23 neutral genes (10 kb) in M.×domestica, M. sieversii, and M. sylvestris (Table 6), suggesting the existence of strong selection on the cafs allele.

TABLE 6
Nucleotide polymorphism for Malus species
at miRNA172p and at 23 neutral genes.

| Species | sequence | N^{a} | S^{b} | π^{c} |
|---|---|---|---|---|
| M. × domestica | cafs^{e} | 19 | 27 | 0.158 |
| M. sieversii | cafs | 15 | 33 | 0.242 |
| M. orientalis | cafs | 15 | 23 | 0.207 |
| M. sylvestris | cafs | 15 | 29 | 0.162 |
| M. baccata | CAFS^{d} | 12 | 46 | 0.331 |
| M. × domestica | Neutral^{e} | 11 | | 0.380 |
| M. sieversii | Neutral^{e} | 10 | | 0.380 |
| M. sylvestris | Neutral^{e} | 21 | | 0.400 |

| | |
|---|---|
| Unpaired oneside Wilcoxon rank sum test ^{f} | , P = 0.014 |

^{a}N: number of accessions sequenced (Supplementary Table 1 and 3)
^{b}S: number of polymorphic sites
^{c}π: the average number of nucleotide differences per site between sequences ⁽²⁶⁾, values are π × 10².
^{d}4 kb sequences of the cafs or CAFS allele of miRNA172p
^{e}10 kb concatenated sequences of 23 neutral genes ⁽²⁷⁾
^{f}Wilcoxon rank sum test between the group of four cafs and the group of CAFS and neutral gene sequences.

In the four species with large fruit, all tested accessions are cafs homozygous (Table 3) and the fixation of the cafs allele in these species indicates that the selection occurred prior to the split of M.×domestica from the other three species. The timing of the split between the four large fruit species is estimated between 20 and 80 thousand years ago based on nuclear DNA analysis⁽²⁸⁾, or even more than one million years ago based on chloroplast DNA sequence information⁽⁹⁾, both of which are much earlier than the estimated commencement of apple domestication, approximately 5000 years ago⁽²⁹⁾. Standard neutral model tests used for analysing domestication loci were not significant for the CAFS locus (Table 7), also suggesting that the beneficial variant, cafs, pre-existed as a common neutral polymorphism prior to domestication, such that positive selection footprints have been erased.

TABLE 7
Standard neutral model test^{a}

| | | Tajima's | Fu and | Fu and |
|---|---|---|---|---|
| Species | N | D | Li's D | Li's F |
| Malus × domestica | 19 | −0.77217 | −0.76933 | −0.89467 |
| Malus sieversii | 15 | −0.386 | −0.84318 | −0.82447 |
| Malus orentalis | 15 | 0.62014 | −0.08782 | 0.12398 |
| Malus sylvestris | 15 | −1.213182 | −0.41212 | −0.72998 |
| Above four species pooled | 64 | −1.28438 | −2.28688 | −2.27538 |
| Malus baccata | 12 | −0.84384 | −0.43042 | −0.61454 |
| Above five species pooled | 76 | −0.79813 | −0.76706 | −0.93562 |

^{a}Tajima's D (²¹⁾, Fu and Li's D* and F * ⁽²²⁾ tests were performed using the 4 kb cafs/CAFS region in the five Malus species. None of the tests were significant, P > 0.05.

In conclusion, the applicants have demonstrated that miRNA172 regulates fruit size in apple. A TE insertion in miRNA172p is strongly associated with reduction of its expression and an increase in fruit size that had been selected by large mammals, before being further strengthened by human selection. The applicant's findings are important for increasing the understanding of the domestication processes of perennial fruits and for enabling the selection for fruit size at the seedling stage in breeding programs for introgression of agronomically important genes from crabapple populations into large domesticated apple.

### Methods

### Production and Molecular Analysis of Apple Transgenic Plants.

To over-express miRNA172 in apple, a plant transformation vector was constructed by transferring the cDNA of the primary transcript of miRNA172p (pri-miRNA172p) ⁽³⁾ (GenBank Accession No EG999280) in Bluescript SK into the BamH1/XhoI sites in pART7 ⁽¹⁰⁾ between the CaMV35S promoter and ocs terminator in sense orientation and then moving the CaMV35S-promoter-miRNA172-cDNA-ocs-terminator fragment from pART7 into the NotI site in pART27 ⁽¹⁰⁾ that also contains the plant selection marker gene NPTII conferring kanamycin resistance. Using this vector, RG apple transgenic plants were produced employing Agrobacterium-mediated plant transformation and kanamycin selection as previously described ^{(11,12)}. The transgenic plants were grown alongside non-transgenic RG plants in a containment glasshouse. Flowers were pollinated with ‘Granny Smith’ pollen.

The transgenic status of the plants was confirmed by PCR analysis of genomic DNA using two primers binding to the NPTII gene⁽¹¹⁾. The presence of a transgenic copy of miRNA172p was ascertained by PCR employing primer 35SF2 (5′-GCACAGTTGCTCCTCTCAGA-3′—SEQ ID NO:45) that binds to the CaMV35S-promoter and primer R4 (FIG. 6) that binds to the miRNA172 cDNA.

Small RNA was extracted from young expanding leaves and opening flowers using the NucleoSpin miRNA kit (Macherey-Nagel). The process included an on-column removal of genomic DNA using DNase. Small RNA was quantified using a Nanodrop ND-1000 spectrophotometer (Thermo Fisher Scientific). The relative levels of miRNA172 were analysed using a stem-loop RT-PCR miRNA assay⁽¹³⁾ with primers designed against miRNA172 and two reference control genes miRNA156 and miRNA159 as previously described⁽¹²⁾. The primers used detect miRNA172 expressed from all miRNA172 genes.

### Tissue Preparation, Staining and Image Analysis.

To analyse the hypanthium and fruit cortex tissue width and cell size, tissue sections (10-μm thickness) of ovaries at full-bloom and fruit at 2 and 5 weeks following pollination and at mature stage were prepared from RG, TRG3 and ‘Aotea’ using the method described previously⁽¹⁵⁾. The sections were dewaxed in xylene, stained in 0.05% (w/v) toluidine blue (pH 4.5) and photographed using a Vanox AHT3 light microscope (Olympus, Tokyo). Hypanthium and cortex tissue width and cell area were measured using ImageJ software (http://imagej.nih.gov/ij/).

### DNA Sequence Analyses.

To determine the DNA sequence diversity at the miRNA172p locus, DNA fragments (up to 3957 bp) were PCR amplified using primers F1 (5′-GTACGCAGTAGAAAGGCCACATGA-3′—SEQ ID NO:46) located in the promoter of miRNA172 and primer R3 (FIG. 6) located in the 3′ end of pri-miRNA172 from 76 accessions of five Malus species (FIG. 9). Primer design was based on the ‘Gold Delicious’ apple genome sequence⁽²⁷⁾. These Malus accessions were collected from different regions of the world to ensure a good representation of each species and had been used in previous studies to determine the genetic contributions of wild species to the cultivated apple⁽²⁸⁾. The sequence diversity data at 23 neutral genetic loci from 42 accessions of three Malus species were taken from a previous publication⁽²⁷⁾ and used to compare with cafs allale sequence diversity in order to determine the cafs allele is under selection (Table 6).

Platinum Taq DNA Polymerase High Fidelity (Invitrogen) was utilized in PCR to minimise DNA synthesis errors. The amplicon was treated with Exonuclease I and Shrimp Alkaline Phosphatase (New England BioLabs) before dispatch to Macrogen (Korea) for sequencing. Sequence assembly and alignment and genetic tree construction were performed using Geneious v6.1.6 (www.geneious.com/). DNA nucleotide diversity and selection tests were performed using DnaSP v5.10.01 (http://www.ub.edu/dnasp/).

Allelotyping of the miRNA172p locus in Malus accessions.

To genotype the miRNA172p locus, PCR amplification was performed using primers F6 and R4 (FIG. 6), located up-stream and down-stream of the TE insertion respectively. The amplification resulted a 331 bp DNA fragment from the CAFS allele of miRNA172p containing no TE insertion and a 494 bp DNA fragment from the cafs allele containing a 154 bp TE and a 9 bp duplication of the insertion site.

Association Analysis of the Cafs Allele with Apple Fruit Size.

The association between miRNA172p alleles and fruit weight was analysed using 159 progeny from a cross between RG and A689-24 (Table 4). A689-24 is a fourth generation descendant from a cross between M.×domestica and M. zumi.

### Quantitative Trait Locus (QTL) Mapping.

The genetic marker for miRNA272p was included in the dataset used to construct the ‘Royal Gala’×A689-24 genetic map⁽¹⁹⁾ using 173 seedlings. Joinmap v3.0 was used to construct the genetic map with a LOD score of 5 for grouping and the Kosambi mapping function to calculate the genetic map distances. QTL analysis was performed using average fruit weight data from 2006, 2007 and 2008 using the A689-24 genetic map for LG11 including the CAFS marker. Interval Mapping was performed and the 95% and 99% QTL intervals were represented as the genetic map regions above and below the maximum LOD score with two and one LOD unit drops, respectively.

### Quantitative RT-PCR.

To determine whether the cafs allele induces a lower miRNA172p expression than the CAFS allele, quantitative RT-PCR analyses were performed using primers F5b and R7 (FIG. 6), that bind specifically to pri-miRNA172p, thereby avoiding any possible interference from miR172a-o. Total RNA was isolated from pooled 1-week-old fruit (n>5) of two CAFS/cafs accessions and four cafs/cafs accessions using a method developed for pine tree RNA extraction ⁽³⁰⁾, and analysed using an Agilent 2100 bioanalyzer (Agilent Co, Ltd, USA) to determine RNA concentration and integrity, then treated with DNase. For each RNA sample, 1 μg RNA was used for cDNA synthesis using the Quantitect® Reverse Transcription Kit (Qiagen) according to the instructions of the manufacturer. Using the cDNA as template, qRT-PCR reactions were carried out using Actin and EF-1a as reference control genes in a LightCycler® 480 (Roche Diagnostics) following previously described procedures⁽²³⁾.

### Summary of Examples

The data presented in the examples above clearly demonstrates the applicability of the applicant's invention showing that when miRNA172 expression is decreased, fruit size is increased. Alternatively, when miRNA172 expression is increased, fruit size is decreased.

The applicant's invention therefore provides valuable new and inventive methods and materials useful for producing (by genetic modification or traditional beeding approaches) fruit of the desired altered size.

### REFERENCES

1. Miller, A. J. & Gross, B. L. From forest to field: perennial fruit crop domestication. American Journal of Botany 98, 1389-1414 (2011).
2. Xia, R., Zhu, H., An, Y. Q., Beers, E. P. & Liu, Z. R. Apple miRNAs and tasiRNAs with novel regulatory networks. Genome Biology 13, R47 (2012).
3. Gleave, A. P. et al. Identification and characterisation of primary microRNAs from apple (Malus domestica cv. Royal Gala) expressed sequence tags. Tree Genetics and Genomes 4, 343-358 (2008).
4. Mlotshwa, S., Yang, Z., Kim, Y. & Chen, X. Floral patterning defects induced by Arabidopsis APETALA2 and microRNA172 expression in Nicotiana benthamiana. Plant Molecular Biology 61, 781-793 (2006).
5. Aukerman, M. J. & Sakai, H. Regulation of flowering time and floral organ identity by a microRNA and its APETALA2-like target genes. Plant Cell 15, 2730-2741 (2003).
6. Harada, T., Kurahashi, W., Yanai, M., Wakasa, Y. & Satoh, T. Involvement of cell proliferation and cell enlargement in increasing the fruit size of Malus species. Scientia Horticulturae 105, 447-456 (2005).
7. Phipps, J. B., Robertson, K. R., Smith, P. G. & Rohrer, J. R. A checklist of the subfamily Maloideae (Rosaceae). Canadian Journal of Botany 68, 2209-2269 (1990).
8. Devoghalaere, F. et al. A genomics approach to understanding the role of auxin in apple (Malus×domestica) fruit size control. BMC Plant Biology 12, (13 Jan. 2012) (2012).
9. Nikiforova, S. V., Cavalieri, D., Velasco, R. & Goremykin, V. Phylogenetic Analysis of 47 Chloroplast Genomes Clarifies the Contribution of Wild Species to the Domesticated Apple Maternal Line. Molecular Biology and Evolution 30, 1751-1760 (2013).
10. Gleave, A. P. A versatile binary vector system with a T-DNA organisational structure conducive to efficient integration of cloned DNA into the plant genome. Plant Molecular Biology 20, 1203-1207 (1992).
11. Yao, J. L., Cohen, D., Atkinson, R., Richardson, K. & Morris, B. Regeneration of transgenic plants from the commercial apple cultivar Royal Gala. Plant Cell Reports 14, 407-412 (1995).
12. Yao, J., Tomes, S. & Gleave, A. P. Transformation of apple (Malus×domestica) using mutants of apple acetolactate synthase as a selectable marker and analysis of the T-DNA integration sites. Plant Cell Reports 32, 703-714 (2013).
13. Varkonyi-Gasic, E., Wu, R., Wood, M., Walton, E. F. & Hellens, R. P. Protocol: a highly sensitive RT-PCR method for detection and quantification of microRNAs. Plant Methods 3(2007).
14. Varkonyi-Gasic, E., Gould, N., Sandanayaka, M., Sutherland, P. & MacDiarmid, R. M. Characterisation of microRNAs from apple (Malus domestica ‘Royal Gala’) vascular tissue and phloem sap. BMC Plant Biology 10, (4 Aug. 2010) (2010).
15. Jackson, D. In situ hybridisation in plants. in Molecular Plant Pathology: A Practical Approach (eds. Gurr, S., McPherson, M. & Bowles, D.) 163-174 (IRL Press, Oxford, 1992).
16. Chen, X. M. A microRNA as a translational repressor of APETALA2 in Arabidopsis flower development. Science (Washington) 303, 2022-2025 (2004).
17. Yant, L. et al. Orchestration of the floral transition and floral development in Arabidopsis by the bifunctional transcription factor APETALA2. Plant Cell 22, 2156-2170 (2010).
18. Jofuku, K. D., Omidyar, P. K., Gee, Z. & Okamuro, J. K. Control of seed mass and seed yield by the floral homeotic gene APETALA2. Proceedings of the National Academy of Sciences of the United States of America 102, 3117-3122 (2005).
19. Chagne, D. et al. Development of a set of SNP markers present in expressed genes of the apple. Genomics 92, 353-358 (2008).
20. Sonia Hamza and Yves Chupeau. (1993) Re-evaluation of conditions for plant regeneration and agrobacterium-mediated transformation from tomato (lycopersicon esculentum). J. Exp. Bot 44: 1837-1845
21. Tajima, F. Statistical method for testing the neutral mutation hypothesis by DNA polymorphism. Genetics 123, 585-95 (1989).
22. Fu, Y. X. & Li, W. H. Statistical tests of neutrality of mutations. Genetics 133, 693-709 (1993).
23. Drummond, R. S. M. et al. Petunia hybrida CAROTENOID CLEAVAGE DIOXYGENASE7 is involved in the production of negative and positive branching signals in petunia. Plant Physiology 151, 1867-1877 (2009).
24. Cornille, A., Giraud, T., Smulders, M. J. M., Roldan-Ruiz, I. & Gladieux, P. The domestication and evolutionary ecology of apples. Trends in Genetics 30, 57-65 (2014).
25. Han, Y. P. & Korban, S. S. Spring: a novel family of miniature inverted-repeat transposable elements is associated with genes in apple. Genomics (San Diego) 90, 195-200 (2007).
26. Tajima, F. Evolutionary relationship of DNA sequences in finite populations Genetics 105, 437-460 (1983).
27. Velasco, R. et al. The genome of the domesticated apple (Malus×domestica Borkh.). Nature Genetics 42, 833-839 (2010).
28. Cornille, A. et al. New insight into the history of domesticated apple: secondary contribution of the European wild apple to the genome of cultivated varieties. PLoS Genetics 8, e1002703 (2012).
29. Juniper, B. E. M., D. J. The story of the apple, (Timber Press, Inc., 2006).
30. Chang, S. J., Puryear, J. & Cairney, J. A simple and efficient method for isolating RNA from pine trees. Plant Molecular Biology Reporter 11, 113-116 (1993).

1-31. (canceled)

32. A method for at least one of:
a) altering the size of a fruit produced by a plant,
a) producing fruit of altered size, and
b) producing a plant with fruit of altered size,
the method comprising altering expression, or activity, of an miRNA172 in a plant that produces the fruit by genetic modification to affect at least one of a) to c), wherein:
i) the plant is from a species in which fruit flesh is derived from hypanthium tissue,
ii) when the expression or activity of the miRNA172 is increased, the fruit size is decreased,
iii) when the expression, or activity, of the miRNA172 is decreased, and the fruit size is increased, and
iv) the altered expression, activity or size is relative to the plant or fruit without the genetic modification.

33. The method of claim 32 wherein expression, or activity, of the miRNA172 is increased by transforming the plant with a polynucleotide encoding the miRNA172.

34. The method of claim 33 wherein the polynucleotide encoding the miRNA172 is operably linked to a promoter sequence.

35. The method of claim 34 wherein the promoter is heterologous with respect to the polynucleotide encoding the miRNA172.

36. A method for detecting at least one of:
a) altered expression of at least one miRNA172,
b) altered expression of at least one miRNA172 gene,
c) presence of a marker associated with altered expression of at least one miRNA172, and
d) presence of a marker associated with altered expression of at least one miRNA172 gene,
wherein:
i) the plant is from a species in which fruit flesh is derived from hypanthium tissue,
ii) increased expression or activity of the miRNA172 indicates that the fruit size will be decreased,
iii) decreased expression or activity of the miRNA172 indicates that the fruit size will be increased, and
iv) the altered, increased or decreased expression or size is relative to that in a wild-type plant.

37. The method of claim 36 wherein any of a) to d) indicates that the plant will produce fruit of altered size.

38. The method of claim 36 which includes the additional step of cultivating the identified plant in which at least one of a) to d) is detected.

39. The method of claim 36 which includes the additional step of breeding from the identified plant in which at least one of a) to d) is detected.

40. A plant, or fruit from the plant, comprising a construct for increasing the expression of at least one miRNA172 or miRNA172 gene in the plant or fruit, wherein the plant is from a species in which fruit flesh is derived from hypanthium tissue, and wherein the fruit size is decreased relative to the fruit from the plant in the absence of the construct.

41. The plant or fruit of claim 40 wherein the construct contains a promoter sequence operably linked to a sequence encoding the miRNA172.

42. The plant or fruit of claim 41 in which the promoter in the construct is heterologous with respect to the sequence encoding the miRNA172.

43. A plant, or fruit from the plant, comprising a construct for reducing or eliminating expression or activity of at least one miRNA172 or miRNA172 gene in a plant, wherein the plant is from a species in which fruit flesh is derived from hypanthium tissue, and wherein the fruit size is increased relative to the fruit from the plant in the absence of the construct.

44. The plant or fruit of claim 43 wherein the construct contains a promoter sequence operably linked to at least part of a miRNA172 gene.

45. The plant or fruit of claim 44 wherein the part of the gene is in an antisense orientation relative to the promoter sequence, and forms part of a hair-pin construct for use in RNAi silencing.

46. The plant or fruit of claim 43 wherein the construct includes a promoter linked to a sequence encoding a mutated target site (target mimic) of miRNA172.

47. The plant or fruit of claim 46 in which the target mimic, includes at least one mismatch relative to the target endogenous miRNA172.

48. The plant or fruit of claim 43 in which the construct is an artificial miRNA-directed anti-miRNA construct.

49. A method for producing a plant that produces at least one fruit of altered size, the method comprising crossing any one of:
a) a plant with altered expression or activity of an miRNA172,
b) a plant comprising a construct for increasing the expression of at least one miRNA172 or miRNA172 gene, and
c) a plant comprising a construct for reducing or eliminating expression or activity of at least one miRNA172 or miRNA172gene,
with another plant, wherein the off-spring produced by the crossing is a plant that produces at least one fruit of altered size.

50. The method of claim 49 wherein the plant is from a species in which fruit flesh is derived from hypanthium tissue, and wherein the expression of the miRNA172 is increased, and the fruit size is decreased.

51. The method of claim 49 wherein the plant is from a species in which fruit flesh is derived from hypanthium tissue, and wherein the expression of the miRNA172 is decreased, and the fruit size is increased.
